(19) United States
(12) Patent Application Publication
(10) Pub. No.: US 2026/0295293 A1
(43) Pub. Date: Oct. 1, 2026

(54) THERAPY DEVICE FOR STIMULATING HAIR GROWTH

(71) Applicant: SHENZHEN NUON MEDICAL EQUIPMENT CO., LTD., Shenzhen (CN)

(72) Inventors: ALAIN DIJKSTRA, Amstelveen (NL); LI XIANG, Shenzhen (CN); Tang Yuanzhou, Shenzhen (CN); Huang Jianlong, Shenzhen (CN); Zhu Yongqi, Shenzhen (CN); Deng Xuanhao, Shenzhen (CN)

(73) Assignee: SHENZHEN NUON MEDICAL EQUIPMENT CO., LTD., Shenzhen (CN)

(21) Appl. No.: 19/629,026

(22) Filed: Mar. 26, 2026

(30) Foreign Application Priority Data
Mar. 27, 2025 (CN) 2025103809938
Mar. 27, 2025 (CN) 2025205632406
Apr. 11, 2025 (CN) 2025206888015
Jan. 21, 2026 (CN) 2026200829817

(51) Int. Cl. A61N 5/06; A61M 11/00; A61M 35/00; A61N 5/067

(52) U.S. Cl. CPC A61N 5/0617; A61M 11/005; A61M 35/003; A61N 5/062; A61N 5/067; A61M 2205/051; A61M 2205/13; A61M 2205/36

(57) ABSTRACT

The present invention relates to a therapy device for stimulating hair growth, comprising a main body having a shell, a liquid storage container, and a treatment interface. The device integrates a phototherapy component with light-emitting elements and a light-transmitting structure having comb-like guide posts for directing therapeutic light toward the scalp. A liquid dispensing system, including medication tubes or an atomizing mechanism, delivers liquid or mist to the scalp. The device further includes a negative pressure mechanism and a laser optical component for enhancing penetration. A dual chamber container enables selective delivery of disinfectant and medication, thereby improving treatment efficiency, absorption, and user convenience while maintaining controlled dispensing, structural stability, and safe operation.

### FIELD OF INVENTION

The present invention relates generally to the field of medical and cosmetic treatment devices and more particularly to a therapy device for stimulating hair growth. Specifically, the invention pertains to a multifunctional scalp treatment apparatus that integrates laser-based therapy, negative-pressure generation, and controlled liquid delivery for enhanced transdermal absorption of therapeutic agents and hair regeneration.

### BACKGROUND

Hair loss and scalp disorders, such as androgenetic alopecia and alopecia areata, are increasingly prevalent worldwide. Traditional treatment methods generally rely on topical medications, oral drugs, or clinical procedures performed in medical institutions. Among these methods, topical medications such as minoxidil solutions are widely used due to their relative simplicity and non-invasive nature. However, the outermost layer of the scalp, the stratum corneum, functions as a barrier that significantly limits the penetration of topical drugs into deeper layers of the skin and hair follicles. As a result, the absorption efficiency of many topical treatments remains low, which reduces their therapeutic effectiveness.

To improve drug absorption, several auxiliary treatment techniques have been developed to enhance the effectiveness of drug delivery. While such techniques significantly improve drug delivery, they typically require professional equipment and trained personnel in hospitals or specialized clinics. The procedures also involve multiple sequential steps, including disinfection, micro-injury treatment, and subsequent drug application, making the process complex, time-consuming, and impractical for frequent home use.

Separately, phototherapy devices utilizing red light, blue light, or near-infrared light have been developed to stimulate scalp circulation and promote hair follicle activity. Phototherapy has also been shown to enhance the absorption of topical medications when applied simultaneously with drug delivery. However, existing phototherapy devices generally function independently of medication delivery systems, requiring users to apply medication and phototherapy as separate steps using separate devices.

In addition, atomization or mist-based delivery systems have been developed to distribute liquid hair-care solutions more evenly over the scalp, improving coverage and utilization compared to direct liquid application. Other applicators use mechanical structures such as medication tubes or comb teeth to deliver medication directly to the scalp upon contact. However, these delivery mechanisms typically lack integration with phototherapy or other treatment modalities that could enhance drug absorption.

Despite these individual developments, existing solutions generally address only a single aspect of scalp treatment. Laser microporation devices focus on creating microchannels, but do not integrate negative pressure stabilization or automated liquid delivery. Phototherapy devices provide light stimulation but lack controlled medication dispensing. Atomization devices improve liquid distribution but do not incorporate phototherapy or microporation to enhance drug penetration. As a result, users must employ multiple devices or undergo separate treatment steps to achieve comprehensive scalp care, which increases treatment complexity, reduces convenience, and limits overall therapeutic effectiveness.

Accordingly, there is a need for a multifunctional therapy device that integrates multiple treatment modalities, including phototherapy, controlled liquid delivery, negative pressure generation, laser microporation, and atomized spraying, into a single apparatus. Such a device should be compact, easy to operate, suitable for home use, and capable of improving drug penetration, distribution, and therapeutic stimulation simultaneously.

### OBJECTS OF THE INVENTION

Some of the objects of the present invention are as follows:

An object of the present invention is to provide a therapy device for stimulating hair growth that integrates phototherapy, controlled liquid delivery, and a delivery enhancement mechanism within a single apparatus, thereby enabling coordinated treatment of a scalp region.

Another object of the present invention is to provide a therapy device comprising a delivery enhancement mechanism that includes at least one of a negative pressure generation mechanism configured to generate a reduced pressure within a treatment cavity, or a mist generation mechanism configured to convert a treatment liquid into a mist before delivery, thereby enhancing penetration, absorption, or distribution of the treatment liquid into the scalp.

Another object of the present invention is to provide a therapy device comprising a laser optical component configured to direct focused laser energy toward a treatment opening to form a plurality of microchannels in the scalp, and a negative pressure generation mechanism configured to cooperate with a liquid dispensing system to facilitate penetration of the treatment liquid through the microchannels into the scalp.

A further object of the present invention is to provide a therapy device comprising a mist generation mechanism and a phototherapy component configured to operate in coordination to improve the distribution and absorption of an atomized treatment liquid on the scalp.

Another object of the present invention is to provide a therapy device comprising a plurality of medication tubes in fluid communication with a liquid storage chamber, wherein each medication tube includes a movable ball bearing configured to seal the tube outlet under normal conditions and to open upon contact with the scalp.

Another object of the present invention is to provide a therapy device comprising a light-transmitting element having a plurality of light guide posts arranged as comb teeth, the light guide posts being configured to guide emitted light from a phototherapy component toward the scalp during use.

A further object of the present invention is to provide a therapy device comprising a push mechanism including a pusher and a sealing head, wherein actuation of the pusher displaces the sealing head to open a connection port between a liquid storage chamber and a liquid distribution chamber, thereby enabling controlled transfer of treatment liquid for atomization.

Another object of the present invention is to provide a therapy device comprising a dual-chamber liquid storage system configured to separately store a disinfectant and a therapeutic medication, along with independent pipelines and valve assemblies, thereby enabling sequential disinfection and treatment without cross-contamination.

Another object of the present invention is to provide a therapy device comprising a contact sensor and a circuit board configured to conditionally control activation of the negative pressure generation mechanism based on detection of a sealing engagement between the device and the scalp.

Yet another object of the present invention is to provide a therapy device having a compact, portable, and user-friendly configuration suitable for home use, thereby reducing reliance on professional clinical procedures while improving therapeutic effectiveness and safety.

### SUMMARY OF THE INVENTION

According to a first aspect of the invention, a therapy device for stimulating hair growth, comprising: a main body defining a treatment cavity and a treatment opening configured to be placed in contact with a scalp; a phototherapy component disposed on or within the main body and configured to direct light energy toward the treatment opening; a liquid dispensing system comprising a liquid storage chamber and at least one liquid outlet in fluid communication with the treatment cavity and configured to deliver a treatment liquid into the treatment cavity; and a delivery enhancement mechanism configured to enhance penetration, absorption, or distribution of the treatment liquid into the scalp, wherein the delivery enhancement mechanism comprises at least one of a negative pressure generation mechanism configured to generate a reduced pressure within the treatment cavity or a mist generation mechanism configured to convert the treatment liquid into a mist prior to delivery, and wherein the phototherapy component and the delivery enhancement mechanism are configured to operate in coordination to improve delivery of the treatment liquid to the scalp.

In one embodiment of the invention, the delivery enhancement mechanism comprises both the negative pressure generation mechanism and the mist generation mechanism.

In one embodiment of the invention, the liquid dispensing system comprises at least one of: a push-actuated dispensing mechanism, a movable ball dispensing mechanism, or an air vent-based dispensing mechanism.

In one embodiment of the invention, the device further comprises a contact sensor configured to detect contact between the treatment opening and the scalp and to control operation of the negative pressure generation mechanism based on detected contact.

In one embodiment of the invention, the device further comprises a flexible sealing member disposed around the treatment opening and configured to form an airtight seal with the scalp.

According to a second aspect of the invention, a therapy device for stimulating hair growth, comprising: a main body defining a treatment cavity and a treatment opening configured to be placed in contact with a scalp; a negative pressure generation mechanism in fluid communication with the treatment cavity and configured to generate a reduced pressure within the treatment cavity; a laser optical component configured to direct laser energy toward the treatment opening; a liquid dispensing system configured to deliver a treatment liquid toward the scalp, wherein the laser optical component is configured to focus laser energy to form a plurality of microchannels in the scalp, and wherein the negative pressure generation mechanism and the liquid dispensing system are configured to cooperate with the microchannels to facilitate penetration of the treatment liquid into the scalp.

In one embodiment of the invention, the laser optical component is configured to receive laser energy from an external laser source through an optical path interface.

In one embodiment of the invention, the laser optical component comprises an internal laser source disposed within the main body.

In one embodiment of the invention, the laser optical component comprises a reflector configured to redirect laser energy and a focusing lens configured to focus the laser energy toward the treatment opening.

In one embodiment of the invention, the focusing lens is positioned at or adjacent to a laser exit opening through which the laser energy exits toward the treatment cavity.

In one embodiment of the invention, the liquid dispensing system comprises at least one spray nozzle configured to deliver the treatment liquid into the treatment cavity.

In one embodiment of the invention, the liquid dispensing system and the laser optical component are configured to deliver treatment liquid and laser energy through a single integrated port opening into the treatment cavity.

In one embodiment of the invention, the laser optical component is aligned along an optical axis passing through the treatment cavity toward the treatment opening.

In one embodiment of the invention, the negative pressure generation mechanism is configured to draw a portion of the scalp into the treatment cavity before laser irradiation and liquid delivery.

According to a third aspect of the invention, a therapy device for stimulating hair growth, comprising: a main body defining a treatment cavity and a treatment opening configured to be placed in contact with a scalp; a phototherapy component configured to direct light energy toward the treatment opening; a liquid dispensing system comprising a liquid storage chamber, a liquid outlet, and a fluid conduit extending between the liquid storage chamber and liquid outlet; and a mist generation mechanism configured to convert the treatment liquid into a mist before delivery into the treatment cavity, wherein the phototherapy component and the mist generation mechanism are configured to operate in coordination to improve distribution and absorption of the treatment liquid on the scalp.

In one embodiment of the invention, the mist generation mechanism comprises an atomizing plate configured to atomize the treatment liquid into fine droplets.

In one embodiment of the invention, the liquid dispensing system further comprises a liquid distribution chamber having a smaller volume than the liquid storage chamber, a connection port connecting the liquid storage chamber and the liquid distribution chamber, a sealing head configured to seal the connection port, and a pusher configured to move the sealing head to open the connection port such that a predetermined amount of liquid is transferred from the liquid storage chamber to the liquid distribution chamber.

In one embodiment of the invention, the device further comprises a heating element configured to heat the treatment liquid before atomization or delivery.

In one embodiment of the invention, the mist generation mechanism comprises an atomizing chamber and an atomizing plate positioned between the atomizing chamber and the treatment cavity.

In one embodiment of the invention, the heating element is positioned upstream of the mist generation mechanism such that the treatment liquid is heated before atomization.

In the context of the specification, when an element is referred to as being “fixed to” or “disposed to” another element, it may either be directly on another element or indirectly on that other element. When a component is said to be “connected” or “connected to” another component, it may be directly connected to another component or indirectly connected to other components on the piece.

In the context of the specification, the terms “first”, “second,” and “third” are only used for descriptive purposes and do not imply the relative importance or implicitly indicate the quantity of technical features indicated.

In the context of the specification, the term “plurality” means two or more than two, unless otherwise indicated.

In the context of the specification, the term “several” means more than one, unless otherwise specified.

In the context of the specification, the term “phototherapy component” refers to any device configured to emit therapeutic light for skin treatment, pain relief, or wellness applications.

In the context of the specification, the term “stimulation component” refers broadly to any element, module, or structure configured to apply a therapeutic or cosmetic stimulus to a user's skin or tissue. Stimulation component may include, but are not limited to, a phototherapy component, a microcurrent component, a Peltier component, a vibrational component, a thermal component, an ultrasonic wave therapy component, a magnetotherapy component, electrical stimulation component, a galvanic component, a TENS component, an RF component, a pulsed electromagnetic field (PEMF) component or a combination thereof.

In the context of the specification, the term “phototherapy element” encompasses any light-emitting device capable of emitting light of therapeutic wavelength(s), including but not limited to light-emitting diodes (LEDs), organic LEDs (OLEDs), laser diodes, or equivalent optical sources. The light may include ultraviolet, visible, near-infrared, or far-infared spectra.

In the context of the specification, the term “shell” refers to the structural enclosure of the device that defines a treatment cavity and a treatment opening. The shell includes a handle portion, head, top cover, bottom cover, or other segments, and is made from polymeric, metallic, composite, or other suitable materials. The main body is a broader assembly that includes the shell along with other components such as the liquid storage container and internal functional elements.

In the context of the specification, the term “control or circuit board” encompasses any printed circuit board (PCB), flexible circuit, or equivalent substrate that supports and electrically connects components of the device, including power supplies, control chips, drivers, or stimulation elements.

In the context of the specification, the term “user” or “subject” is intended to broadly cover humans, animals, or other recipients of the treatment, unless otherwise specifically limited.

In the context of the specification, the term “delivery enhancement mechanism” refers to any component or system configured to enhance penetration, absorption, or distribution of a treatment liquid into the scalp. The delivery enhancement mechanism comprises at least one of a negative pressure generation mechanism configured to generate a reduced pressure within a treatment cavity, or a mist generation mechanism configured to convert the treatment liquid into a mist before delivery. In some embodiments, the delivery enhancement mechanism comprises both the negative pressure generation mechanism and the mist generation mechanism.

In the context of the specification, the term “LED module” refers to one or more light-emitting diode (LED) elements that are electrically connected and configured to emit light of specific wavelengths suitable for therapeutic purposes. The LED module may include drive circuitry, heat dissipation structures, and optical elements such as lenses or diffusers to control light distribution.

In the context of the specification, the term “light source” or “phototherapy source” etc. refers to a source emitting coherent laser light, or light-emitting diodes (“LEDs”). The term “light therapy” refers to light generated from any of the sources, such as lasers, LED sources, or Super luminous diodes (“SLD”).

In the context of the specification, “Light Emitting Diodes (LEDs)” refer to semiconductor diodes capable of emitting electromagnetic radiation when supplied with an electric current. The LEDs are characterized by superior power efficiencies, smaller sizes, rapid switching speeds, physical robustness, and longer lifespans compared to incandescent or fluorescent lamps. The one or more LEDs may include through-hole type LEDs (generally emitting electromagnetic radiation in red, green, yellow, blue, and white colors), Surface Mount Technology (SMT) LEDs, Bi-color LEDs, Pulse Width Modulated RGB (Red-Green-Blue) LEDs, and high-power LEDs, among others.

Materials used in one or more LEDs may vary from one embodiment to another, depending upon the frequency of radiation required. Different frequencies can be obtained from LEDs made from pure or doped semiconductor materials. Commonly used semiconductor materials include nitrides of Silicon, Gallium, Aluminum, Boron, Zinc Selenide, etc., in pure form or doped with elements such as Aluminum and Indium. For example, red and amber colors are produced from Aluminum Indium Gallium Phosphide (AlGaInP) based compositions, while blue, green, and cyan use Indium Gallium Nitride based compositions. White light may be produced by mixing red, green, and blue lights in equal proportions, while varying proportions may be used to generate a wider color gamut. White and other colored lightings may also be produced using phosphor coatings such as Yttrium Aluminum Garnet (YAG) in combination with a blue LED to generate white light, and Magnesium-doped potassium fluorosilicate in combination with a blue LED to generate red light.

In addition to conventional mineral-based LEDs, one or more LEDs may also be provided on an Organic LED (OLED) based flexible panel or an inorganic LED-based flexible panel. Such OLED panels may be generated by depositing organic semiconducting materials over Thin Film Transistor (TFT) based substrates. Further, a discussion on the generation of OLED panels can be found in Bardsley, J. N (2004), “International OLED Technology Roadmap”, IEEE Journal of Selected Topics in Quantum Electronics, Vol. 10, No. 1, that is included herein in its entirety, by reference. An exemplary description of flexible inorganic light-emitting diode strips can be found in granted U.S. Pat. No. 7,476,557 B2, titled “Roll-to-roll fabricated light sheet and encapsulated semiconductor circuit devices”, which is included herein in its entirety by reference.

In the context of this specification, terms like “light”, “radiation”, “irradiation”, “emission” and “illumination”, etc. refer to electromagnetic radiation in frequency ranges varying from the Ultraviolet (UV) frequencies to Infrared (IR) frequencies and wavelengths, wherein the range is inclusive of visible light, UV and IR frequencies and wavelengths. It is to be noted here that UV radiation can be categorized in several ways depending on respective wavelength ranges, all of which are envisaged to be under the scope of this invention. For example, UV radiation can be categorized as Hydrogen Lyman-α (122-121 nm), Far UV (200-122 nm), Middle UV (300-200 nm), and Near UV (400-300 nm). The UV radiation may also be categorized as UVA (400-315 nm), UVB (315-280 nm), and UVC (280-100 nm). Similarly, IR radiation may also be categorized into several categories according to respective wavelength ranges, which are again envisaged to be within the scope of this invention. A commonly used subdivision scheme for IR radiation includes Near IR (0.75-1.4 μm), Short-Wavelength IR (1.4-3μm), Mid-Wavelength IR (3-8 μm), Long-Wavelength IR (8-15 μm), and Far IR (15-1000 μm).

Unless otherwise stated, the term “light” as used in this specification encompasses electromagnetic radiation in the visible (380-780 nm) and infrared (780 nm-1000 nm) ranges, particularly red light (620-750 nm) and near-infrared (750-1400 nm) wavelengths commonly used in photobiomodulation therapy. Particular wavelengths which may be selected as the dominant emissive wavelength may include the following, without any preference to be indicated by order: 400 nm, 405 nm, 420 nm, 430 nm, 450 nm, 465 nm, 515 nm, 530 nm, 532 nm, 590 nm, 630 nm, 633 nm, 640 nm, 650 nm, 655 nm, 660 nm, 670 nm, 680 nm, 780 nm, 785 nm, 810 nm, 830 nm, 840 nm, 850 nm, 860 nm, 870 nm, 904 nm, 915 nm, 980 nm, 1015 nm, 1060 nm, 1065 nm, 1070 nm, 1200 nm, and 1400 nm. As used herein, the term “light therapy” refers to the use of one or more light sources of any type that emit light with a wavelength between about 400 and 1400 nm. The device may also emit blue or ultraviolet light for surface-level treatments such as acne reduction or microbial control.

### BRIEF DESCRIPTION OF THE ACCOMPANYING DRAWINGS

The accompanying drawings illustrate the best mode for carrying out the invention as presently contemplated and set forth hereinafter. The present invention may be more clearly understood from a consideration of the following detailed description of the preferred embodiments taken in conjunction with the accompanying drawings, wherein like reference letters and numerals indicate the corresponding parts in various figures in the accompanying drawings, and in which:

FIG. 1 shows a top perspective view of a therapy device for stimulating hair growth, in accordance with an embodiment of the present invention.

FIG. 2 shows a bottom perspective view of the therapy device for stimulating hair growth, in accordance with an embodiment of the present invention.

FIG. 3 illustrates a cross-sectional view of the therapy device for stimulating hair growth, in accordance with an embodiment of the present invention.

FIG. 4 illustrates an enlarged cross-sectional view of section A given in FIG. 3 of the therapy device for stimulating hair growth, in accordance with an embodiment of the present invention.

FIG. 5 shows a top perspective view of a mounting bracket of the therapy device for stimulating hair growth, in accordance with an embodiment of the present invention.

FIG. 6 shows a cross-sectional view of another configuration of a therapy device for stimulating hair growth, in accordance with an embodiment of the present invention.

FIG. 7 shows a cross-sectional view of a therapy device for stimulating hair growth, shown in FIG. 6, showing part A and part B, in accordance with an embodiment of the present invention.

FIG. 8 shows an enlarged view of part A of FIG. 7, in accordance with an embodiment of the present invention.

FIG. 9 shows an enlarged view of part B of FIG. 7, in accordance with an embodiment of the present invention.

FIG. 10 shows an exploded view of the configuration of the therapy device for stimulating hair growth given in FIG. 6, in accordance with an embodiment of the present invention.

FIG. 11 shows an exploded view of a push component and a sealing plug of the therapy device for stimulating hair growth, in accordance with an embodiment of the present invention.

FIG. 12 illustrates a bottom view of the therapy device for stimulating hair growth, showing a pair of conductive electrodes, a fan, and a mist outlet, in accordance with an embodiment of the present invention.

FIG. 13 shows a perspective view of yet another configuration of a therapy device for stimulating hair growth, in accordance with an embodiment of the present invention.

FIG. 14 shows a bottom view of a treatment opening of the therapy device for stimulating hair growth, in accordance with an embodiment of the present invention.

FIG. 15 shows a cross-sectional view of the configuration of the therapy device for stimulating hair growth, given in FIG. 13, in accordance with an embodiment of the present invention.

FIG. 16 shows a side view of the therapy device for stimulating hair growth, given in FIG. 13, without a shell, in accordance with an embodiment of the present invention.

FIG. 17 shows a schematic diagram of an optical path of the laser through a reflector and a focus lens, in accordance with an embodiment of the present invention.

FIG. 18 shows an exploded view of the configuration of the therapy device for stimulating hair growth given in FIG. 13, in accordance with an embodiment of the present invention.

### DETAILED DESCRIPTION OF THE INVENTION

Embodiments of the present invention disclosure will be described more fully hereinafter with reference to the accompanying drawings in which like numerals represent like elements throughout the figures, and in which example embodiments are shown.

The detailed description and the accompanying drawings illustrate the specific exemplary embodiments by which the disclosure may be practiced. These embodiments are described in detail to enable those skilled in the art to practice the invention illustrated in the disclosure. It is to be understood that other embodiments may be utilized, and other changes may be made, without departing from the spirit or scope of the present disclosure. The following detailed description is therefore not to be taken in a limiting sense, and the scope of the present invention disclosure is defined by the appended claims. Embodiments of the claims may, however, be embodied in many different forms and should not be construed as limited to the embodiments set forth herein.

The terms “a” and “an” herein do not denote a limitation of quantity, but rather denote the presence of at least one of the referenced items. The terms “having”, “comprising”, “including”, and variations thereof signify the presence of a component.

Embodiments of the present invention provide a therapy device for stimulating hair growth comprising a main body having a shell and a liquid storage container, a plurality of medication tubes, a phototherapy component, a light-transmitting element, and a delivery enhancement mechanism. The main body is provided with a liquid storage container having a liquid storage chamber configured to store a therapeutic liquid and a plurality of mounting holes for accommodating the medication tubes. Each medication tube is in fluid communication with the liquid storage chamber to facilitate controlled delivery of the therapeutic liquid to the scalp surface. The delivery enhancement mechanism is configured to enhance penetration, absorption, or distribution of the therapeutic liquid into the scalp.

The phototherapy component is disposed within the main body and includes one or more light-emitting elements configured to emit light of predetermined wavelengths. The light-transmitting element comprises an integrally formed mounting portion and a plurality of light guide columns, wherein the mounting portion is fitted onto the main body and includes through holes for receiving the medication tubes. The light guide columns are arranged corresponding to the phototherapy component to guide emitted light toward the scalp.

The device further includes a liquid storage container, a circuit board 162, and a battery 160, wherein the phototherapy component and associated components are electrically connected to the circuit board 162. The arrangement enables simultaneous delivery of liquid medication and light therapy to the scalp.

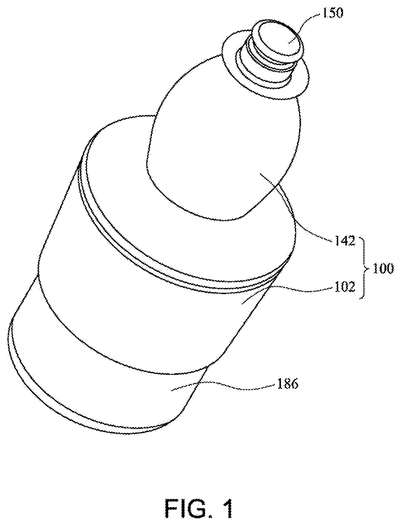

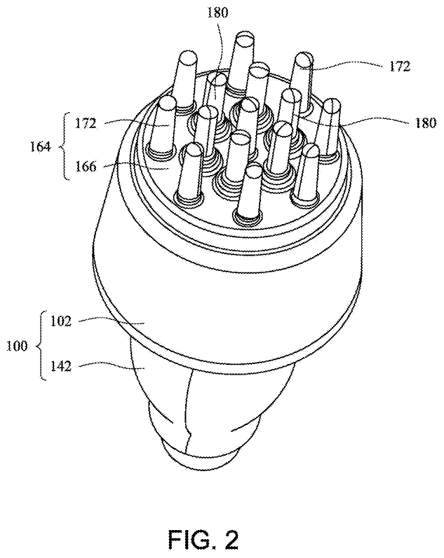

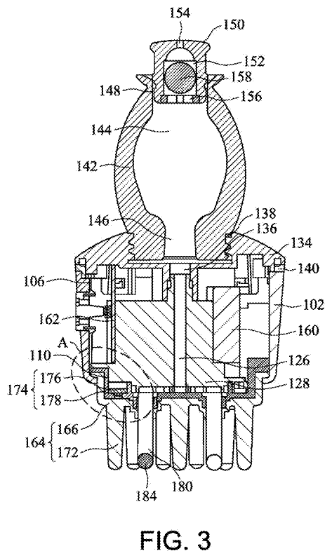

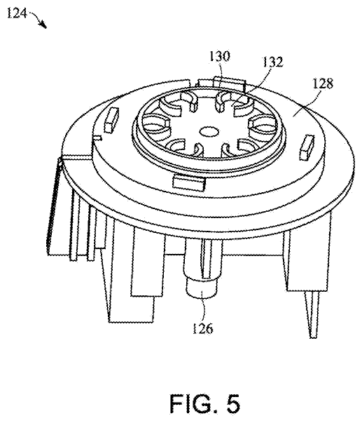

Referring to FIGS. 1 to 5, the present invention relates to a therapy device for stimulating hair growth. The therapy device for stimulating hair growth comprises a main body 100 that includes a shell 102 and a liquid storage container 142. The shell 102 defines a treatment cavity and a treatment opening configured to be placed in contact with a scalp. The shell 102 further comprises a light-transmitting element 164, a phototherapy component 174, and a plurality of medication tubes 180. The liquid storage container 142 defines a liquid storage chamber 144 that is configured to store a medicinal liquid. The plurality of medication tubes 180 is disposed within the shell 102 and is in fluid communication with the liquid storage chamber 144, thereby allowing the medicinal liquid to be delivered to a scalp treatment area.

The light-transmitting element 164 is arranged within the main body 100 and comprises a plurality of light guide posts 172, which are a plurality of comb teeth configured to guide therapeutic light toward the scalp. The light guide posts 172 are spaced apart from the medication tubes 180 and arranged to correspond to the phototherapy component 174 such that light emitted by the phototherapy component 174 is efficiently transmitted through the light guide posts 172.

In this embodiment, each medication tube 180 is provided with a ball bearing 184 disposed within the interior of the medication tube 180. A portion of the ball bearing 184 extends outward from an end of the medication tube 180 to seal the outlet of the medication tube 180 under normal conditions. When the device is pressed against the scalp during use, the ball bearing 184 is pushed inwardly into the medication tube 180 due to contact with the scalp, thereby opening the outlet of the medication tube 180 and allowing the medicinal liquid stored in the liquid storage chamber 144 to flow outwardly through the medication tube 180 and onto the scalp.

Each light guide post 172 is positioned adjacent to a corresponding medication tube 180 and extends substantially parallel thereto. When the medication tube 180 contacts the scalp, the distal end of the light guide post 172 is likewise configured to contact or closely approach the scalp surface. The light guide post 172 is formed from a transparent or light-transmissive material to enable efficient transmission of light.

In an embodiment, the therapy device for stimulating hair growth can include one or more stimulation components that may include, but are not limited to, a phototherapy component, a microcurrent component, a Peltier component, a vibrational component, a thermal component, an ultrasonic wave therapy component, a magnetotherapy component, electrical stimulation component, a galvanic component, a TENS component, an RF component, a pulsed electromagnetic field (PEMF) component or a combination thereof. In one configuration, comb teeth are provided with microcurrent electrodes to provide microcurrent therapy.

The phototherapy component 174 comprises one or more light-emitting elements 178 configured to emit light having a predetermined wavelength, such as red light, blue light, or violet light, suitable for scalp treatment. When the phototherapy component 174 is activated, light emitted by the light-emitting elements 178 is transmitted through the corresponding light guide posts 172 and delivered to the scalp, thereby providing phototherapy simultaneously with the application of medication.

In certain embodiments, the therapy device further includes a battery 160 disposed within the main body 100 for supplying electrical power to the phototherapy component 174. Alternatively, the phototherapy component 174 may be powered through an external power supply connected to the device during operation.

The light-emitting elements 178 are configured as red light sources, such that red-light therapy is provided during operation. The red light therapy can facilitate improved penetration and absorption of the applied medicinal liquid into the scalp tissue, thereby enhancing the therapeutic effectiveness of the medication.

The light guide posts 172 are distributed around the periphery of the medication tubes 180. In such a configuration, the medication tubes 180 are arranged in a central region, while the light guide posts 172 are distributed in an outer peripheral region surrounding the medication tubes 180. This arrangement allows the medication tubes 180 to be more concentrated in the central region, thereby facilitating more precise medication application while simultaneously providing a relatively large phototherapy coverage area. As a result, during operation, phototherapy can be continuously applied to the treatment area regardless of the direction in which the device is moved, thereby improving therapeutic effectiveness. In alternative embodiments, the light guide posts 172 and the medication tubes 180 can be arranged in an alternating configuration.

In an embodiment, the phototherapy component 174 includes a ring-shaped lamp plate 176, and the light-emitting elements 178 are disposed on the ring-shaped lamp plate 176. The corresponding light guide posts 172 are arranged to align with the ring-shaped lamp plate 176 such that light emitted from the light-emitting elements 178 can be efficiently coupled into the respective light guide posts 172. In certain configurations, the light-emitting elements 178 correspond one-to-one with the light guide posts 172. This structural configuration enables the phototherapy component 174 to be formed as an integrated module, thereby facilitating convenient assembly and installation within the device. The number of light-emitting elements 178 can be greater than, less than, or equal to the number of light guide posts 172. In other configurations, a single light-emitting element 178 can correspond to multiple light guide posts 172, or certain light-emitting elements 178 can be positioned between adjacent light guide posts 172.

In an embodiment, the light-transmitting element 164 further includes a mounting portion 166, on which the plurality of light guide posts 172 are disposed. The mounting portion 166 is configured to be mounted on the main body 100. The mounting portion 166 is provided with a plurality of first through holes 168, which are spaced apart from the light guide posts 172 and configured to receive the medication tubes 180. In particular, the mounting portion 166 covers a portion of the main body 100 on which the medication tubes 180 are installed, and each medication tube 180 extends through the corresponding first through hole 168. This configuration increases the contact area between the light-transmitting element 164 and the main body 100, thereby improving structural stability. Furthermore, the medication tubes 180 passing through the first through holes 168 can restrict the movement of the light-transmitting element 164, thereby further enhancing the installation stability of the light-transmitting element 164.

In an embodiment, the shell 102 is provided with a plurality of mounting holes 112, each mounting hole 112 being configured to receive and support a corresponding medication tube 180. During assembly, the medication tubes 180 are first installed into the corresponding mounting holes 112 of the shell 102. Subsequently, the light-transmitting elements 164 are accommodated into the shell 102 such that the plurality of medication tubes 180 passes through the corresponding plurality of first through holes 168. This assembly arrangement facilitates accurate alignment of the structural components and improves the overall structural integrity and reliability of the device.

In an embodiment, the light guide post 172 and the mounting portion 166 are integrally formed as a single structural component. In such embodiments, the light-transmitting element 164 may be formed entirely as a flexible component, for example, by integrally molding the light-transmitting element 164 from an elastic material such as soft rubber or a similar elastomeric material. This integral flexible structure provides a stable connection between the light guide posts 172 and the mounting portion 166 while reducing the number of assembly steps required during manufacturing. The light-transmitting element 164 can instead be formed as a rigid component manufactured from a rigid transparent or semi-transparent material.

Further, each medication tube 180 is configured to be tightly fitted within the corresponding first through hole 168 of the mounting portion 166. In particular, before installation of the light-transmitting element 164, the inner diameter of the first through hole 168 can be slightly smaller than the outer diameter of the medication tube 180. During assembly, when the medication tube 180 is inserted into the first through hole 168, the mounting portion 166 undergoes elastic deformation such that the wall of the first through hole 168 presses against the outer surface of the medication tube 180. As a result, a tight interference fit is formed between the medication tube 180 and the first through hole 168. This configuration not only improves the installation stability of the mounting portion 166 but also enhances the sealing performance between the medication tube 180 and the corresponding mounting hole 112, thereby reducing the possibility of medicinal liquid leakage.

In an embodiment, the shell 102 includes a bottom cover 110. The shell 102 includes a first opening 104, and the bottom cover 110 is arranged to close or cover the first opening 104. The medication tubes 180 are mounted on the bottom cover 110, while the mounting portion 166 of the light-transmitting element 164 is arranged on the outer side of the bottom cover 110. During assembly, the medication tubes 180 can first be installed onto the bottom cover 110. Subsequently, the light-transmitting element 164 is mounted such that the medication tubes 180 extend through the corresponding first through holes 168. Thereafter, the assembled module, including the medication tubes 180, the bottom cover 110, and the light-transmitting element 164, can be mounted onto the shell 102. This modular assembly arrangement facilitates coordinated installation of the medication tubes 180 and the light-transmitting element 164 and improves installation reliability. In an alternative embodiment, the shell 102 and the bottom cover 110 can be integrally formed as a single structure.

In an embodiment, the phototherapy component 174 is disposed within the bottom cover 110. The bottom cover 110 is provided with a light-transmitting portion corresponding to the phototherapy component 174 to allow light emitted by the phototherapy component 174 to pass therethrough. The bottom cover 110 includes a light-transmitting hole 114, or the corresponding portion of the bottom cover 110 can be formed from a transparent or light-transmissive material.

The shell 102 further includes a first annular portion 108 surrounding the first opening 104, and the bottom cover 110 includes a second annular portion 116 disposed around its periphery. The outer diameter of the second annular portion 116 is larger than the inner diameter of the first opening 104 and is positioned inside the shell 102 when assembled. The periphery of the mounting portion 166 is further provided with an annular limiting portion 170 configured to abut against the first annular portion 108 and the second annular portion 116. Specifically, the outer diameter of the annular limiting portion 170 is greater than the inner diameter of the first opening 104 so that the annular limiting portion 170 is positioned inside the shell 102 during assembly. The annular limiting portion 170 abuts against the inner side of the first annular portion 108, while the second annular portion 116 abuts against the opposite side of the annular limiting portion 170. Through this arrangement, the light-transmitting element 164 can be securely fixed by a clamping action formed between the shell 102 and the bottom cover 110 on the annular limiting portion 170.

Such a configuration allows the outer surface of the light-transmitting element 164 to remain smooth and uninterrupted, thereby improving both sealing performance and the overall appearance of the device. Furthermore, when the light-transmitting element 164 is formed as a flexible component, the annular limiting portion 170 can additionally function to seal the gap between the shell 102 and the bottom cover 110, thereby improving the sealing effect while reducing the need for additional sealing rings. In alternative embodiments, the mounting portion 166 can also be fixed to the bottom cover 110 by bonding or adhesive attachment.

In an embodiment, the shell 102 further comprises a mounting frame 124. The mounting frame 124 includes a conduit 126 and a cover plate 128 disposed at one end of the conduit 126. The cover plate 128 cooperates with the bottom cover 110 to define a transfer cavity 122 therebetween. One end of the conduit 126 is configured to communicate with the liquid storage chamber 144, while the other end communicates with the transfer cavity 122. The medication tubes 180 are further configured to communicate with the transfer cavity 122. In particular, the mounting frame 124 is arranged within the shell 102. At least one of the cover plate 128 and the bottom cover 110 is provided with a groove such that when the cover plate 128 abuts against the bottom cover 110, the groove cooperates with the opposing surface to form the transfer cavity 122. In use, the medicinal liquid delivered through the conduit 126 enters the transfer cavity 122 and is subsequently distributed to the plurality of medication tubes 180. This arrangement ensures that the medicinal liquid is supplied to each of the medication tubes 180 while maintaining a relatively simple structural configuration. Furthermore, when the cover plate 128 abuts against the bottom cover 110, the second annular portion 116 can press against the annular limiting portion 170, thereby reducing or eliminating the need for additional fastening structures, such as screws, between the annular limiting portion 170, the first annular portion 108, and the second annular portion 116. As a result, the overall structure of the therapy device for stimulating hair growth can be simplified.

In an embodiment, the end of each medication tube 180 located within the shell 102 is provided with a limiting protrusion 182. The outer diameter of the limiting protrusion 182 is larger than the diameter of the corresponding mounting hole 112. The cover plate 128 is further provided with a locking rib 130 configured to abut against the limiting protrusion 182. During assembly, the medication tube 180 is inserted into the mounting hole 112 from the interior side of the bottom cover 110. After installation, the limiting protrusion 182 abuts against an edge of the mounting hole 112, thereby preventing the medication tube 180 from being withdrawn from the mounting hole 112. Additionally, the locking rib 130 abuts against the limiting protrusion 182 to further restrict movement of the medication tube 180 and ensure that the medication tube 180 remains securely installed.

The locking rib 130 can be formed as an annular rib configured to abut against the limiting protrusion 182, and the locking rib 130 can further include a notch 132. Alternatively, a plurality of locking ribs 130 may be provided and distributed at intervals along the circumferential direction corresponding to the limiting protrusion 182. Such configurations allow the limiting protrusion 182 to be subjected to substantially uniform circumferential forces, thereby improving installation stability of the medication tube 180 while also preventing obstruction of the normal flow of the medicinal liquid.

In some embodiments, the bottom cover 110 is provided with a mounting annular groove 118 surrounding the transfer cavity 122. The phototherapy component 174 is disposed within the mounting annular groove 118. The bottom wall of the mounting annular groove 118 is provided with the light-transmitting hole 114, thereby allowing light emitted by the phototherapy component 174 to pass therethrough. This configuration facilitates the positioning and installation of the phototherapy component 174 while effectively limiting its movement.

In an embodiment, the bottom cover 110 further includes an annular rib 120, wherein an inner region defined by the annular rib 120 and a cover surface of the bottom cover 110 forms the transfer cavity 122, while an outer region defined by the annular rib 120 forms the mounting annular groove 118. Through this arrangement, the annular rib 120 effectively separates the transfer cavity 122 from the mounting annular groove 118, thereby resulting in a simplified and well-organized structural configuration of the bottom cover 110.

The main body 100 further includes a top cover 134 and the liquid storage container 142. The shell 102 includes a second opening 106 positioned opposite the first opening 104. The top cover 134 is mounted at the second opening 106 and has a second through hole 136. The liquid storage container 142 is detachably mounted on a side of the top cover 134 facing away from the bottom cover 110. The liquid storage container 142 includes the liquid storage chamber 144 and a liquid outlet 146 in communication with the liquid storage chamber 144. The liquid outlet 146 is configured to communicate with the second through hole 136 of the top cover 134. This detachable configuration facilitates convenient removal and installation of the liquid storage container 142 for purposes such as cleaning, refilling, or replacement.

In an embodiment, the top cover 134 can be connected to the shell 102 through a fastening structure such as snap-fit engagement, adhesive bonding, or threaded fastening. The end of the mounting frame 124, located away from the bottom cover 110, can abut against the top cover 134. In this manner, the mounting frame 124 can press the bottom cover 110 into position such that the second annular portion 116 presses against the annular limiting portion 170, thereby contributing to the fixation of the light-transmitting element 164 and further simplifying the structural design of the therapy device.

In some embodiments, the top cover 134 is provided with a mounting groove 138, and the second through hole 136 is formed on a bottom wall of the mounting groove 138. A peripheral wall of the mounting groove 138 is provided with an internal thread, while an end of the liquid storage container 142 having the liquid outlet 146 is provided with a corresponding external thread configured to mate with the internal thread. Through this threaded engagement, the liquid storage container 142 can be securely screwed into the mounting groove 138 of the top cover 134. This threaded installation ensures reliable mounting of the liquid storage container 142 and reduces the risk of the liquid storage container 142 detaching or leaking during use.

Optionally, the liquid storage container 142 may be formed from a transparent or translucent material, thereby enabling a user to visually observe the quantity of medicinal liquid remaining within the liquid storage container 142 in real time, which facilitates convenient use and timely replenishment of the medicinal liquid.

In an embodiment, the shell 102 further includes a light-transmitting ring 140 and at least one decorative light-emitting element. The shell 102 is provided with the second opening 106 positioned opposite the first opening 104, and the top cover 134 is arranged to cover the second opening 106. The light-transmitting ring 140 is disposed between the top cover 134 and the shell 102 and includes an exposed light-transmitting surface. The decorative light-emitting element is disposed within the light-transmitting ring 140 such that, when activated, light emitted by the light-emitting element is transmitted outwardly through the exposed light-transmitting surface of the light-transmitting ring 140. In this manner, the decorative light-emitting element may function as an indicator light or decorative light. The number of decorative light-emitting elements may be one or more. The plurality of decorative light-emitting elements may be distributed circumferentially around the light-transmitting ring 140.

In an embodiment, the device further includes the battery 160 and a circuit board 162, both of which are disposed within the main body 100. The battery 160 and the phototherapy component 174 are electrically connected to the circuit board 162 so that electrical power from the battery 160 can be supplied to the phototherapy component 174 during operation, thereby enabling convenient portable use of the device. In some embodiments, the aforementioned light-emitting element associated with the light-transmitting ring 140 can also be electrically connected to the circuit board 162. The therapy device may further include a charging component electrically connected to the circuit board 162 and configured to allow an external charger to charge the battery 160. The charging component may include, for example, a wireless charging coil, a charging interface, or charging pins, among other suitable charging structures.

In some embodiments, an end of the liquid storage chamber 144 opposite the medication tubes 180 is provided with a container mouth 148. The device further includes a sealing sleeve 150 installed at the container mouth 148. The sealing sleeve 150 includes a receiving cavity 152, a vent hole 154, and a third through hole 156, wherein both the vent hole 154 and the third through hole 156 communicate with the receiving cavity 152. The third through hole 156 is configured to communicate with the liquid storage chamber 144, while the vent hole 154 communicates with the external environment.

A movable ball 158 is disposed within the receiving cavity 152. The movable ball 158 is configured to assume a first state, in which the vent hole 154 is blocked, and a second state, in which the vent hole 154 is open. In certain embodiments, a plurality of third through holes 156 can be provided, and/or the third through holes 156 can be formed as non-circular openings, such as elliptical holes, polygonal holes, or irregularly shaped holes, to prevent the movable ball 158 from obstructing the third through hole 156.

During use, when the therapy device is oriented such that the medication tubes 180 are positioned below the sealing sleeve 150, the movable ball 158 moves under the influence of gravity into the second state, thereby opening the vent hole 154. In this state, the liquid storage chamber 144 communicates with the external environment through the third through hole 156, the receiving cavity 152, and the vent hole 154, thereby allowing the medicinal liquid stored in the liquid storage chamber 144 to flow toward the medication tubes 180 under the action of gravity.

After use, when the therapy device is inverted such that the medication tubes 180 are positioned above the sealing sleeve 150, and the sealing sleeve 150 is positioned below, the movable ball 158 moves under gravity into the first state, thereby sealing the vent hole 154 and preventing the medicinal liquid from flowing out of the liquid storage chamber 144. Additionally, during operation, a user may manually control the flow of the medicinal liquid by selectively covering or uncovering the vent hole 154 with a finger, thereby providing convenient control over the dispensing of the medicinal liquid.

In an embodiment, the receiving cavity 152 of the sealing sleeve 150 is formed as an elongated cavity. The vent hole 154 and the third through hole 156 are respectively disposed at opposite ends along a longitudinal direction of the receiving cavity 152. A movable ball 158 is arranged within the receiving cavity 152 and is configured to move along the longitudinal direction of the receiving cavity 152 between different positions corresponding to the opening or closing of the vent hole 154. Such an elongated configuration allows sufficient movement space for the movable ball 158 while preventing the vent hole 154 from requiring an excessively large radial dimension, thereby contributing to a more compact structural design.

In an embodiment, the device can include an air vent-based dispensing mechanism configured to facilitate controlled discharge of liquid from the liquid storage container 142. The vent hole 154 is arranged to allow ambient air to enter the container to balance internal pressure during liquid dispensing. In addition to or as an alternative to a dedicated valve structure, such as a movable ball, the air vent mechanism may be manually actuated by a user. For example, the user may selectively cover or uncover the vent hole 154 using a finger to regulate airflow into the container, thereby controlling the dispensing rate of the liquid.

Optionally, the sealing sleeve 150 can be detachably mounted at the container mouth 148 of the liquid storage chamber 144. This detachable configuration facilitates convenient removal and reinstallation of the sealing sleeve 150 for purposes such as cleaning, maintenance, replacement, or refilling of the liquid storage chamber 144, thereby improving the practicality and serviceability of the therapy device for stimulating hair growth.

In an embodiment, the device further comprises a protective cover 186. The protective cover 186 is detachably mounted on the main body 100 and is configured to cover the light guide posts 172 and the medication tubes 180. After use of the device, the protective cover 186 can be installed to enclose the light guide posts 172 and the medication tubes 180, thereby effectively protecting these components from contamination, damage, or unintended contact during storage or transportation.

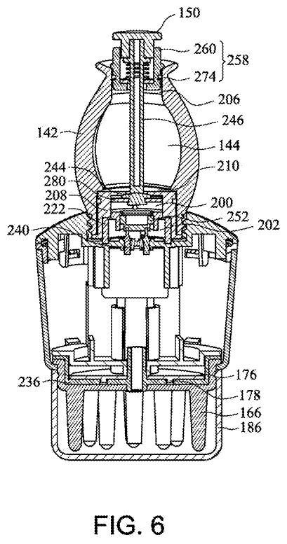

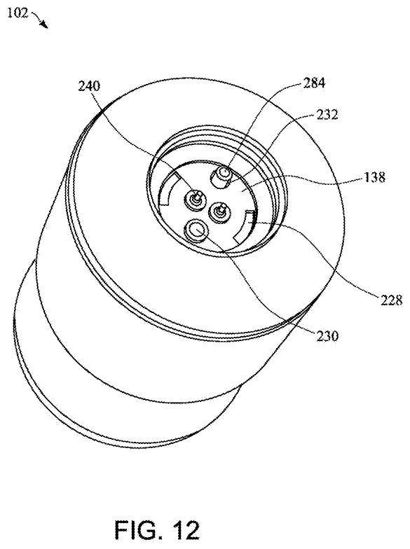

Referring now to FIGS. 6 to 12, an alternate configuration of the therapy device for stimulating hair growth is described, in which a therapy device for stimulating hair growth includes an atomizing mechanism for converting the treatment liquid into a mist.

The therapy device for stimulating hair growth comprises a main body integrating a shell and a liquid storage container. In this configuration, the liquid storage container has a liquid storage chamber, a liquid dispensing chamber, an atomizing chamber, a sealing assembly, a pushing component, and an atomizing structure. The shell defines a fluid pathway including a connection port between the liquid storage chamber and the dispensing chamber, and an installation port between the dispensing chamber and the atomizing chamber.

The sealing assembly includes a sealing head configured to selectively close the connection port, while the pushing component is movably arranged to actuate the sealing head to permit controlled transfer of liquid from the storage chamber to the dispensing chamber. The atomizing structure is disposed at the installation port and is configured to atomize the liquid present in the dispensing chamber into a fine mist.

The atomized liquid is discharged through a mist outlet formed in the atomizing chamber, wherein the comb teeth are arranged around the mist outlet to enable simultaneous combing and distribution of the atomized liquid over hair and scalp. The device further optionally includes a fan, a battery, and a control circuit for facilitating atomization and airflow-assisted dispersion of the mist.

In an alternative embodiment of the present invention, a therapy device for stimulating hair growth is provided that not only performs conventional hair-combing functions but is also configured to cooperate with a hair care solution to improve hair and scalp care. In particular, the hair care solution is discharged in an atomized form, allowing it to be distributed over a relatively larger area of the scalp and hair. This atomized delivery facilitates improved absorption and utilization of the hair care solution, thereby enhancing the overall hair care effect.

Referring to FIGS. 1, 2, 4, 5, and 7, in an alternate configuration, a therapy device for stimulating hair growth includes a main body 100 having a shell 102, a liquid storage container 142, a mounting portion 166, a sealing head 244, a pusher 246, and an atomizing component 252. The mounting portion 166 is fixed to the main body 100 and includes a plurality of light guide posts 172, which are light transmissive comb teeth extending outwardly from the main body 100 for combing hair during use.

In an embodiment, the delivery enhancement mechanism comprises a mist generation mechanism configured to convert the treatment liquid into a mist before delivery into the treatment cavity. The droplets formed by the mist generation mechanism have a size in the range of approximately 1 to 50 microns. The mist generation mechanism may include an atomizing component, such as an atomizing plate, ultrasonic element, or heating-based atomizer, arranged in communication with the liquid dispensing system. The treatment liquid is transformed into fine mist droplets or aerosolized particles, thereby increasing the surface area of the liquid and enabling more uniform distribution across the scalp. The mist form of the treatment liquid improves contact efficiency and facilitates enhanced absorption into the scalp and surrounding follicular regions.

In this embodiment, the liquid storage container 142 defines a liquid storage chamber 144, a liquid distribution chamber 200, and an atomizing chamber 202. A connection port 210 is provided between the liquid storage chamber 144 and the liquid distribution chamber 200. An installation port 188 is provided between the liquid distribution chamber 200 and the atomizing chamber 202. The atomizing chamber 202 further communicates with a mist outlet 230 through which atomized liquid is discharged. The volume of the liquid distribution chamber 200 is smaller than that of the liquid storage chamber 144.

The sealing head 244 is arranged to seal the connection port 210, thereby preventing liquid contained in the liquid storage chamber 144 from entering the liquid distribution chamber 200 under normal conditions. The pusher 246 is movably connected to the liquid storage container 142 and is configured to actuate the sealing head 244.

The device integrates a push-actuated dispensing mechanism including the pusher 246. When the pusher 246 is pressed or pushed, the sealing head 244 is displaced to open the connection port 210. The atomizing component 252 is fixed at the installation port 188 and is configured to atomize the liquid contained within the liquid distribution chamber 200.

During an initial state, the sealing head 244 blocks the connection port 210 so that liquid stored in the liquid storage chamber 144 cannot enter the liquid distribution chamber 200. When atomized spraying is required, the pusher 246 is pressed downward, thereby causing the sealing head 244 to move and open the connection port 210. As a result, liquid contained in the liquid storage chamber 144 flows into the liquid distribution chamber 200 through the connection port 210. Once the liquid distribution chamber 200 is filled with liquid, or the required amount has entered, the sealing head 244 returns to a position that seals the connection port 210 again. Subsequently, the liquid present in the liquid distribution chamber 200 is atomized by the atomizing component 252 to form a fine mist. The mist is discharged through the mist outlet 230 of the atomizing chamber 202 and is applied to the scalp and hair.

On one hand, because the liquid storage chamber 144 has a relatively larger volume while the liquid distribution chamber 200 has a smaller volume, the liquid storage chamber 144 can store a relatively large amount of hair care solution for multiple uses without frequent refilling. Meanwhile, the liquid distribution chamber 200 enables the user to more easily control the amount of liquid dispensed during a single use, thereby reducing waste. Additionally, when the device is not in use, the sealing head 244 seals the connection port 210 so that liquid does not remain in the liquid distribution chamber 200. This configuration reduces the likelihood of leakage from the liquid distribution chamber 200 or the atomizing chamber 202.

On the other hand, because the pusher 246 is provided, the user can easily open the connection port 210 by simply pressing the pusher 246, thereby providing a simple and convenient operation.

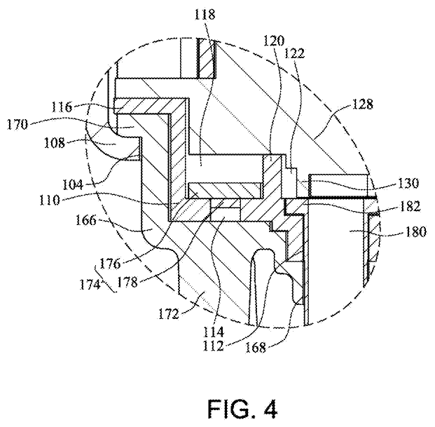

Referring again to FIGS. 4, 6, and 8, in an embodiment, the liquid storage container 142 is further provided with a clearance port 206 communicating with the liquid storage chamber 144. The pusher 246 passes through the clearance port 206, with an upper end of the pusher 246 exposed outside the liquid storage container 142 and a lower end positioned in contact with the sealing head 244. The pusher 246 and the clearance port 206 are sealed together by a sealing assembly 258.

In this configuration, the pusher 246 extends through the liquid storage chamber 144 and abuts against the sealing head 244 so that the pusher 246 can move upward and downward within the liquid storage chamber 144. To prevent leakage between the pusher 246 and an edge of the clearance port 206, the sealing assembly 258 is arranged to provide a sealing function.

The sealing assembly 258 includes a sealing plug 260 fixed to the liquid storage container 142. The sealing plug 260 is provided with a hollow structure through which the pusher 246 passes. To improve sealing performance, the outer peripheral surface of the sealing plug 260 is provided with one or more sealing protrusions 262. The sealing protrusions 262 may be formed as annular ribs surrounding the sealing plug 260 and configured to abut against an inner wall of the main body 100, thereby enhancing the sealing engagement between the sealing plug 260 and the main body 100. Through this arrangement, leakage between the pusher 246 and the main body 100 can be effectively prevented while allowing the pusher 246 to reciprocate relative to the sealing assembly 258 during operation.

In an embodiment, the outer peripheral surface of the sealing plug 260 is further provided with a second annular receiving groove 264. A second sealing ring (not shown) can be disposed within the second annular receiving groove 264 and is configured to abut against the inner surface of the liquid storage container 142. Through this arrangement, the sealing protrusion 262 and the second sealing ring cooperate to form a dual sealing structure between the sealing plug 260 and the liquid storage container 142, thereby further improving the sealing performance of the device.

In alternative embodiments, an adhesive may be provided within the second annular receiving groove 264 so that the sealing plug 260 is fixed to the liquid storage container 142 by adhesive bonding. In such configurations, the sealing plug 260 may be permanently secured to the liquid storage container 142 and cannot be readily removed.

In an embodiment, an inner circumferential surface of the sealing plug 260 is provided with a limiting member 266. The sealing assembly 258 further includes a first sealing ring 274 disposed within the sealing plug 260. The lower end of the first sealing ring 274 abuts against the limiting member 266, and a sealing sleeve 150 of the first sealing ring 274 is arranged around the pusher 246. In an embodiment, the sealing plug 260 is tightly inserted into the connection port 210 and is secured to the liquid storage container 142 through an interference fit, thereby sealing the edge of the connection port 210.

The sealing plug 260 can have a relatively large axial height, with an upper portion of the sealing plug 260 protruding outwardly from the connection port 210 and a lower portion extending into the liquid storage chamber 144. Such a configuration provides a relatively large vertical contact area between the sealing plug 260 and the inner surface of the liquid storage container 142, thereby achieving improved sealing performance and a secure fit. However, because the overlap height between the sealing plug 260 and the pusher 246 is relatively large, direct contact between the entire inner circumferential surface of the sealing plug 260 and the pusher 246 would result in a large contact area and consequently a high frictional force, which could hinder smooth vertical movement of the pusher 246.

The first sealing ring 274 is arranged on the inner circumferential surface of the sealing plug 260 to provide a localized sealing contact with the pusher 246. This configuration reduces the effective contact area between the sealing plug 260 and the pusher 246, thereby lowering frictional resistance and enabling smoother upward and downward movement of the pusher 246. The limiting member 266 functions to axially retain the first sealing ring 274, preventing it from moving downward within the sealing plug 260.

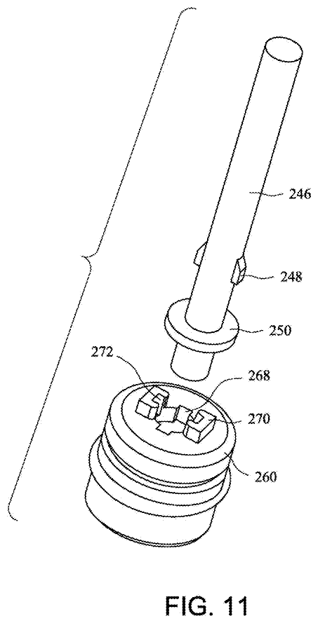

Referring to FIG. 11, a stop protrusion 248 is provided on a side portion of the pusher 246. The stop protrusion 248 is configured to abut against the bottom surface of the sealing plug 260. Specifically, after the pusher 246 moves upward by a predetermined distance, the stop protrusion 248 contacts the bottom surface of the sealing plug 260, and more particularly the bottom surface of the limiting member 266, thereby preventing further upward movement of the pusher 246.

Furthermore, the limiting member 266 is provided with a clearance notch 268 configured to allow passage of the stop protrusion 248. When removal of the pusher 246 is required, the pusher 246 can first be rotated until the stop protrusion 248 aligns with the clearance notch 268. The pusher 246 can then be pulled upward, allowing the stop protrusion 248 to pass through the clearance notch 268 without being blocked by the limiting member 266. After the pusher 246 is removed, liquid can be introduced into the liquid storage chamber 144 through a hollow region defined between the sealing plug 260 and the first sealing ring 274, thereby enabling convenient refilling of the liquid storage chamber 144.

In some embodiments, a bottom surface of the limiting member 266 is further provided with a limiting rib 270 that surrounds and defines a limiting groove 272. A stop protrusion 248 formed on the pusher 246 is configured to be received within the limiting groove 272. The limiting rib 270 functions to restrict rotational movement of the pusher 246, thereby preventing undesired rotation of the pushing member during operation. In addition, the limiting rib 270 provides a guiding effect for the stop protrusion 248 during vertical movement of the pusher 246, thereby reducing the likelihood of lateral displacement and ensuring that the lower end of the pusher 246 can reliably actuate the sealing head 244.

Optionally, the number of stop protrusions 248 can be two, and correspondingly, the number of clearance notches 268 and limiting ribs 270 can also be two, thereby providing balanced engagement and improved structural stability.

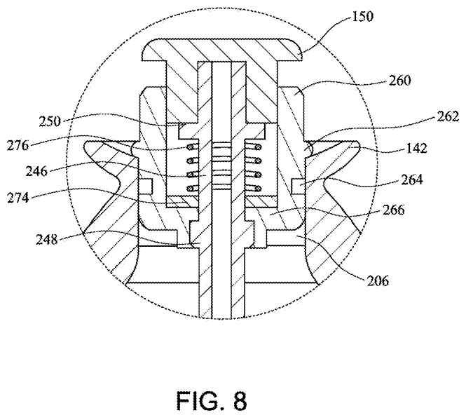

Referring to FIG. 8, the therapy device further includes a sealing sleeve 150. The sealing sleeve 150 is sleeved over an upper end of the pusher 246 and extends into the sealing plug 260, thereby forming a sealing engagement with the inner circumferential surface of the sealing plug 260. In this configuration, the sealing sleeve 150 provides an additional sealing function to prevent leakage between the sealing plug 260 and the pusher 246. Moreover, since the sealing sleeve 150 is arranged over the pusher 246, a user can directly apply pressure to the sealing sleeve 150 to actuate the pusher 246, thereby providing a smoother and more comfortable operating feel.

In an embodiment, a side portion of the pusher 246 is further provided with an abutment protrusion 250 located above the first sealing ring 274. A first return spring 276 is arranged between the abutment protrusion 250 and the first sealing ring 274, and the first return spring 276 is configured to bias the pusher 246 upward. During operation, when the pusher 246 is pressed downward, the first return spring 276 is compressed between the abutment protrusion 250 and the first sealing ring 274. When the external pressing force is released, the first return spring 276 expands to push the pusher 246 upward, thereby returning the pusher 246 to its initial position.

The lower end of the sealing sleeve 150 abuts against the abutment protrusion 250, such that the abutment protrusion 250 also functions to limit the position of the sealing sleeve 150. Additionally, because the first return spring 276 and the first sealing ring 274 are both arranged below the sealing sleeve 150, these components are effectively enclosed within the sealing plug 260 by the sealing sleeve 150. As a result, external water, dust, or contaminants are prevented from entering and contacting the first return spring 276 and the first sealing ring 274.

Moreover, the abutment protrusion 250 can be formed as an annular protrusion surrounding the pusher 246. In an alternative embodiment, multiple abutment protrusions 250 can be arranged circumferentially around the pusher 246.

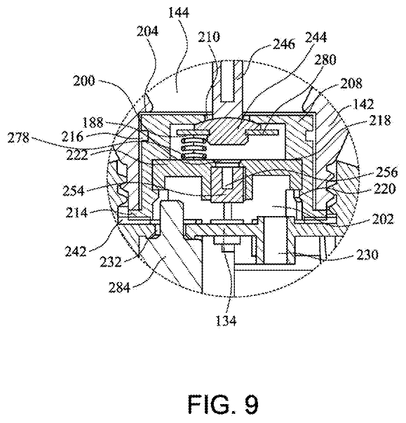

Referring to FIG. 9, a top surface of the sealing head 244 is configured to have a central raised portion and relatively lower peripheral edges, such as an upwardly convex arcuate or umbrella-shaped configuration. The sealing head 244 is disposed within the liquid distribution chamber 200, and when the sealing head 244 seals the connection port 210, the top surface of the sealing head 244 abuts against a surface surrounding the connection port 210 on a side of the liquid distribution chamber 200. The shape of the sealing head 244 allows the sealing head to adapt to connection ports 210 having different dimensions, thereby improving sealing reliability. Even when slight manufacturing tolerances or processing errors occur in the connection port 210, the sealing head 244 can still effectively seal the connection port 210.

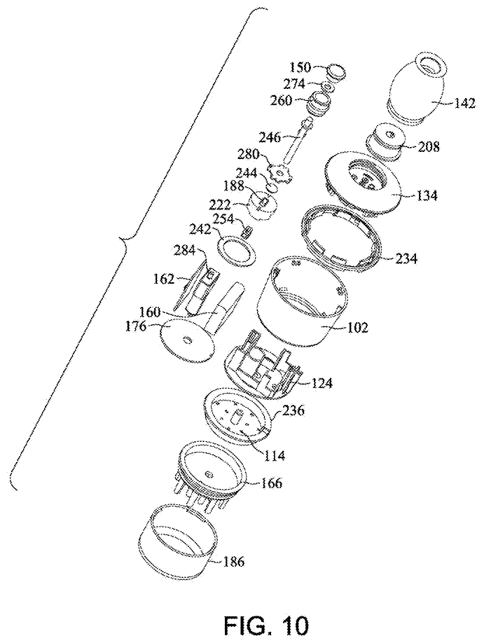

Referring to FIGS. 8 and 10, the therapy device further includes a guide plate 280 fixed to the sealing head 244. The guide plate 280 is provided with at least one guide groove, and a cover base 208 is provided with corresponding guide ribs at the inner surface of the cover base 208, configured to slidably engage the guide groove. Through this sliding engagement, the guide ribs guide the vertical movement of the guide plate 280 when the sealing head 244 moves upward and downward. This guiding structure prevents the sealing head 244 from deviating from its predetermined movement path and ensures that the sealing head 244 can accurately reseal the connection port 210 when returning to its sealing position.

In an embodiment, the sealing head 244 can include a fixing annular groove formed on its circumferential surface. The guide plate 280 can be arranged around the sealing head 244 such that an inner edge of the guide plate 280 is engaged within the fixing annular groove. The sealing head 244 is formed from a relatively elastic sealing material and thus exhibits inherent flexibility. The addition of the guide plate 280 helps ensure that the sealing head 244 moves along a predetermined path during operation. Moreover, because the guide plate 280 surrounds the sealing head 244, the forces acting on the sealing head 244 are distributed substantially uniformly along its circumference, thereby reducing the likelihood of tilting or misalignment of the sealing head 244.

Optionally, the guide plate 280 may include a plurality of guide grooves formed along its edge, while the inner surface of the cover base 208 may include a corresponding plurality of guide ribs, thereby further improving guiding stability during movement of the sealing head 244.

In some embodiments, a second return spring 278 is arranged between the guide plate 280 and a cavity wall of the liquid distribution chamber 200. The second return spring 278 is configured to return the guide plate 280 and the sealing head 244 to a sealing position at the connection port 210. Specifically, when the pusher 246 is pressed downward, the sealing head 244 and the guide plate 280 move downward and compress the second return spring 278. When the external pressing force applied to the pusher 246 is removed, the second return spring 278 elastically expands upward, thereby pushing the guide plate 280 and the sealing head 244 upward so that the sealing head 244 reseals the connection port 210. During this upward movement, the sealing head 244 may simultaneously drive the pusher 246 to return to its original position.

Additionally, two second return springs 278 can be provided and arranged respectively on opposite sides of the guide plate 280, thereby ensuring balanced restoring forces and stable movement of the sealing head 244.

In an embodiment, when the liquid distribution chamber 200 becomes filled with liquid, or when the amount of liquid within the liquid distribution chamber 200 reaches a predetermined level, the sealing head 244 may reseal the connection port 210 through one or more mechanisms. In one implementation, the pusher 246 is structurally connected to the sealing head 244, such that the pusher 246 may be pulled upward to drive the sealing head 244 upward, thereby closing the connection port 210. In another implementation, a reset mechanism, such as the second return spring 278, is arranged below the sealing head 244. When the pusher 246 drives the sealing head 244 downward, the reset mechanism is compressed. Upon removal of the external force, the reset mechanism elastically returns upward, thereby lifting the sealing head 244 and the pusher 246 back to their original positions. To maintain balanced forces at upper and lower portions of the pusher 246 and reduce the likelihood of swinging or misalignment, the first return spring 276 described above may also be provided.

In an embodiment, the sealing assembly 258, the sealing head 244, and the sealing sleeve 150 can each be formed from an elastic sealing material, such as silicone or other elastomeric materials, thereby enhancing sealing reliability.

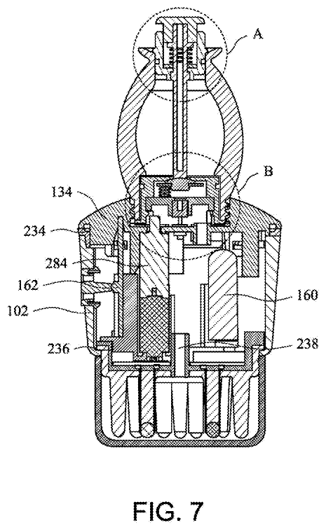

Referring to FIGS. 7 and 9, to facilitate rapid discharge of gas from the atomizing chamber 202 and to reduce the temperature within the chamber to prevent overheating and potential damage to the atomizing component 252, the hair care comb may further include a fan 284. The atomizing chamber 202 is provided with an air outlet 232, and an outlet of the fan 284 is positioned adjacent to the air outlet 232. When the fan 284 is activated, airflow is directed into the atomizing chamber 202 through the air outlet 232, thereby accelerating the discharge of atomized gas from the mist outlet 230.

Referring to FIGS. 6, 7, and 12, in an embodiment, the main body 100 includes a liquid storage container 142 and a shell 102. The shell 102 houses a circuit board 162, a battery 160, and the fan 284. The battery 160 is configured to supply electrical power to the circuit board 162 and the fan 284. The mounting groove 138 is formed on the top surface of the shell 102, into which the lower end of the liquid storage container 142 is inserted.

Specifically, the liquid storage container 142 includes a liquid storage chamber 144, a lower end of which is inserted into the mounting groove 138 and detachably connected to the shell 102. In an embodiment, the liquid storage container 142 can be provided with external threads, while an inner wall of the mounting groove 138 can be provided with internal threads, thereby enabling threaded engagement between the liquid storage container 142 and the shell 102. The liquid storage container 142 and the shell 102 may be connected through a snap-fit structure. The detachable connection between the liquid storage container 142 and the shell 102 allows the liquid storage container 142 to be conveniently removed for cleaning or maintenance.

In an embodiment, the liquid storage container 142 further includes the cover base 208 and a fixing base 222. The liquid storage container 142 is configured with a downward-facing opening. The cover base 208 is fixed to the liquid storage container 142 to seal the lower end of the liquid storage container 142, thereby cooperatively forming the liquid storage chamber 144. The cover base 208 is further provided with the connection port 210. The fixing base 222 is fixed relative to the cover base 208, and a liquid distribution chamber 200 is defined between the cover base 208 and the fixing base 222. The fixing base 222 is provided with an installation port 188 configured to receive the atomizing component 252.

Referring to FIG. 9, an inner wall of the liquid storage container 142 is provided with an annular first stepped surface 204 that faces the mounting groove 138. The cover base 208 is configured with a downward opening, and the lower end of the cover base 208 is provided with a limiting flange 214 extending outwardly. During assembly, the cover base 208 is inserted into the liquid storage container 142 from bottom to top. An upper end of the cover base 208 abuts against the first stepped surface 204, while the limiting flange 214 abuts against a lower end surface of the liquid storage container 142, thereby restricting upward movement of the cover base 208 and securing the assembly in position.

Referring to FIG. 10, to prevent leakage between the cover base 208 and the liquid storage container 142, a first annular receiving groove 216 is formed on an outer circumferential surface of the cover base 208. A sealing ring may be disposed within the first annular receiving groove 216, such that the sealing ring seals and engages with an inner surface of the liquid storage container 142. Through this arrangement, a tight sealing engagement between the cover base 208 and the liquid storage container 142 is achieved, which also helps to limit downward movement of the cover base 208.

Referring again to FIG. 9, a sealing gasket 242 may further be arranged at the bottom portion of the mounting groove 138. The sealing gasket 242 is annular and surrounds the opening of the mounting groove 138. The limiting flange 214 of the cover base 208 abuts against the sealing gasket 242 when the liquid storage container 142 is engaged on the shell 102. This configuration prevents moisture and dust from entering the interior of the device through the interface between the liquid storage container 142 and the shell 102, and also assists in preventing downward displacement of the cover base 208.

In an embodiment, an inner wall of the cover base 208 is provided with a second stepped surface 218 and a limiting boss 220. The second stepped surface 218 is arranged facing downward, while the limiting boss 220 is positioned below the second stepped surface 218. The fixing base 222 is retained between the second stepped surface 218 and the limiting boss 220, thereby securing the fixing base 222 in position within the cover base 208.

In an embodiment, the inner wall of the cover base 208 is further provided with guide ribs. In addition, a mist outlet 230 and the air outlet 232 are formed at the bottom portion of the mounting groove 138.

In an embodiment, the space defined between the fixing base 222 and the bottom of the mounting groove 138 forms the atomizing chamber 202.

Referring to FIG. 10, the bottom of the mounting groove 138 is further provided with a positioning groove 228 and a conductive electrode 240. The fixing base 222 includes a positioning protrusion configured to be received within the positioning groove 228, thereby ensuring proper positioning during assembly. The atomizing component 252 includes a ceramic base 254 and a heating element 256. The heating element 256 is fixed to the bottom surface of the ceramic base 254 and is electrically connected to the conductive electrode 240.

During assembly, the fixing base 222, the cover base 208, and the atomizing component 252 are first assembled within the liquid storage container 142, after which the liquid storage container 142 is threaded into the mounting groove 138. When assembled, the conductive electrode 240 comes into electrical contact with the heating element 256, thereby enabling electrical conduction. To ensure accurate alignment between the conductive electrode 240 and the heating element 256, the positioning protrusion and the positioning groove 228 are provided to achieve reliable positioning.

In an embodiment, a plurality of conductive electrodes 240 is provided, including two positive electrodes and two negative electrodes, thereby ensuring a stable electrical connection with the heating element 256.

The ceramic base 254 may be formed through high-temperature sintering, resulting in a structure containing numerous micropores. Due to surface tension and capillary action, liquid can uniformly penetrate the ceramic base 254 and adhere to its surface. When the heating element 256 is electrically energized, heat generated by the heating element 256 is transferred to the ceramic base 254, thereby heating the liquid absorbed within the ceramic base 254 and causing the liquid to become atomized.

The mounting portion 166 is arranged at the bottom portion of the shell 102, and a spray nozzle 238 is provided at the bottom surface of the shell 102. The spray nozzle 238 is connected to the mist outlet 230 through a conduit or pipe. The mounting portion 166 includes a clearance opening corresponding to the position of the spray nozzle 238, and the plurality of light guide posts 172 are arranged surrounding the clearance opening. In this configuration, the spray nozzle 238 is positioned centrally among the plurality of light guide posts 172, allowing the atomized mist to be more effectively dispersed around each light guide post 172 during operation.

Referring to FIGS. 4 and 8, in an embodiment, the shell 102 includes a top cover 134, a light-transmitting ring 234, and a bottom portion 236, which are sequentially connected from top to bottom. The top cover 134 closes the upper end of the light-transmitting ring 234 and includes the mounting groove 138. The bottom portion 236 closes the lower end of the shell 102.

The shell 102 further includes a mounting frame 124 disposed within the shell 102. The mounting frame 124 includes a plurality of reinforcing ribs that collectively define multiple accommodating grooves. These grooves are configured to respectively receive and support internal components, including the battery 160, the circuit board 162, and the fan 284, thereby ensuring stable positioning and organized arrangement of the internal components within the shell 102.

In an embodiment, the shell 102 further includes the lamp plate 176 and at least one light-emitting element 178. The light-emitting element 178 is mounted on the lamp plate 176 and electrically connected to the circuit board (not shown). The bottom portion 236 is provided with the light-transmitting hole 114 through which light emitted from the light-emitting element 178 can pass.

The mounting portion 166 is disposed at a lower portion of the shell 102 and is formed from a light-transmitting material. The mounting portion 166 covers a lower surface of the bottom portion 236, thereby allowing light emitted from the light-emitting element 178 to pass through the light-transmitting hole 114 and further propagate through the mounting portion 166 to the exterior. In this manner, light can be delivered to a scalp region when the light guide posts 172 are brought into contact with a user's hair or scalp.

In some embodiments, the light-emitting element 178 is configured to emit one or more therapeutic wavelengths, including but not limited to red light, infrared light, blue light, or violet light. These light wavelengths may be used to perform phototherapy, for example, to promote hair growth, stimulate scalp circulation, or reduce inflammation of the scalp.

In addition, a decorative light strip may be arranged within the shell 102. The decorative light strip is disposed around the light-transmitting ring 234, such that light emitted by the decorative light strip is visible externally through the light-transmitting ring 234. The decorative light strip can serve as a visual indicator of device operation and enhance the aesthetic appearance of the hair care comb.

Furthermore, a protective cover 186 is provided at the lower end of the shell 102. The protective cover 186 is detachably mounted on the main body 100 and covers the light guide posts 172 and the medication tubes 180. After use, the protective cover 186 covers the light guide posts 172 and the medication tubes 180, thereby effectively protecting these components from contamination, physical damage, or accumulation of dust and debris during storage or transportation.

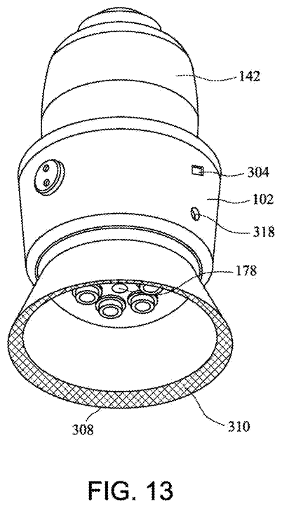

Referring now to FIGS. 13 to 18, a further alternate configuration of the therapy device for stimulating hair growth is described, in which the device includes a laser optical component and a negative pressure generation mechanism.

In an alternate configuration, the therapy device for stimulating hair growth comprises a main body integrated with a shell, a liquid storage container, a delivery enhancement mechanism, a laser optical component, and a liquid spraying mechanism. In this configuration, the delivery enhancement mechanism comprises a negative pressure generation mechanism. The shell 102 defines a treatment cavity having a treatment opening configured to be placed against a scalp surface and further includes an optical path interface for connection with an external laser source.

The negative pressure generation mechanism is arranged in communication with the treatment cavity and is configured to generate a negative pressure environment within the treatment cavity. The laser optical component is disposed within the shell 102 and includes an optical path connected to the optical path interface, and a laser outlet oriented toward the treatment opening for directing laser energy to the scalp.

The liquid spraying mechanism comprises at least one spray nozzle communicating with the treatment cavity for delivering a liquid onto the scalp. The device further includes a liquid supply mechanism having a disinfectant chamber and a medication chamber, selectively connectable to the spray nozzle, along with associated pumps, pipelines, atomizing components, and valve assemblies for controlled liquid delivery.

Furthermore, the device can include a flexible sealing member arranged around the treatment opening, a contact sensor configured to detect contact between the device and the scalp, a circuit board for coordinated operation, and a sterilization lamp disposed within the treatment cavity.

The invention further provides a therapy device for stimulating hair growth utilizing a laser source externally connected or disposed within the device, wherein the external laser source is operatively connected to the optical path interface.

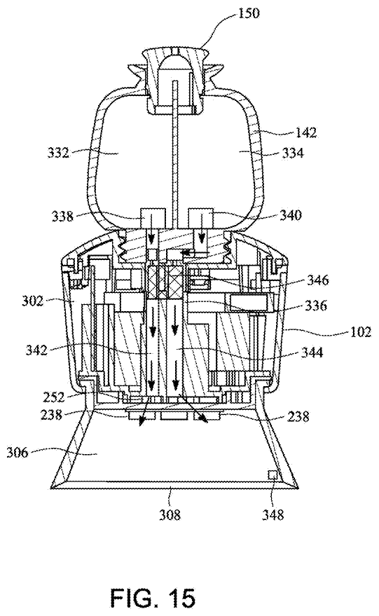

Referring to FIGS. 13 and 15, an alternate configuration of a therapy device for stimulating hair growth is provided, which includes a main body 100, a liquid storage container 142, a shell 102, a negative pressure generation mechanism, a laser optical component 320, and a liquid spraying mechanism.

As shown in FIG. 15, the main body 100 includes a shell 102 that defines a treatment cavity 306 and a treatment opening 308. The treatment opening 308 is configured to be positioned against a scalp region of a user during operation. The main body 100 is further provided with an optical path interface 304 configured for connection with an external laser source.

The interior of the shell 102 forms a mounting cavity 302, and the treatment cavity 306 is disposed below the mounting cavity 302. The mounting cavity 302 is configured to accommodate internal functional components of the device. The optical path interface 304 is arranged within the mounting cavity 302 and exposed to the exterior of the shell 102, allowing detachable connection with the external laser source and providing a transmission channel for laser energy. The treatment cavity 306 is configured as an operational region that directly interacts with the scalp. A lower portion of the treatment cavity 306 is provided with the treatment opening 308, which functions as an access port through which laser irradiation and liquid delivery can be applied to the scalp.

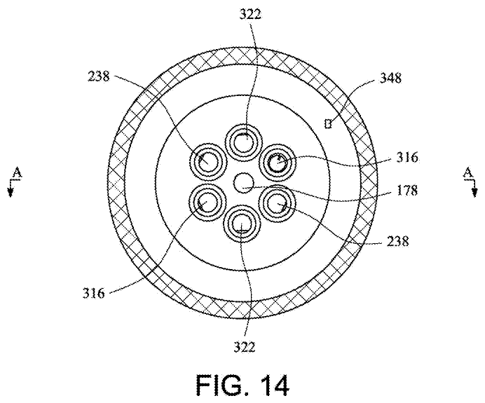

Referring to FIG. 14, in one embodiment, the delivery enhancement mechanism comprises a negative pressure generation mechanism configured to generate a reduced pressure within the treatment cavity. The negative pressure generation mechanism may include a negative pressure generator 314, an air extraction pathway, and one or more suction ports communicating with the treatment cavity. During operation, air is withdrawn from the treatment cavity to create a sub-atmospheric pressure environment, thereby drawing the scalp toward the treatment opening and stabilizing the treatment region. This negative pressure condition enhances penetration of the treatment liquid by promoting inward diffusion and retention of the liquid within the scalp tissue and hair follicle structures. In an embodiment, the negative pressure generation mechanism is disposed within the mounting cavity 302 and communicates with the treatment cavity 306 through a predefined air passage. The negative pressure generation mechanism may include components such as a miniature vacuum pump and associated valves. During operation, the mechanism extracts air from the treatment cavity 306 to create a negative pressure environment therein. The negative pressure assists in drawing and stabilizing a portion of the scalp toward the treatment opening 308, thereby improving fixation of the treatment area and facilitating penetration of medication. The positioning of the negative pressure generation mechanism within the shell 102 is preferably arranged to avoid interference with a laser transmission path.

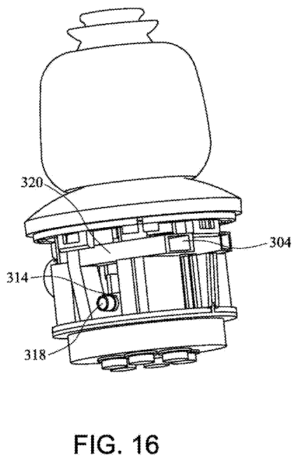

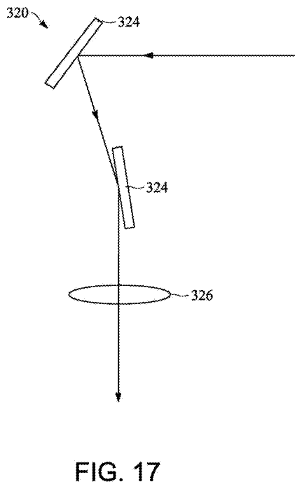

Referring to FIGS. 16 and 17, the laser optical component 320 is disposed within the shell 102. The laser optical component comprises a laser diode and a focusing lens configured to deliver pulse laser energy to form microchannels in the scalp. An optical input path of the laser optical component 320 is connected to the optical path interface 304, and the laser optical component 320 further includes a laser outlet 322 oriented toward the treatment opening 308. In some embodiments, the laser optical component 320 is arranged within the mounting cavity 302. The optical input end of the laser optical component 320 receives laser energy from the external laser source through the optical path interface 304. The laser optical component 320 may include one or more optical elements, such as lenses, mirrors, or beam-shaping components, for directing and adjusting the laser beam path. After optical adjustment, the laser beam is emitted from the laser outlet 322 toward the treatment opening 308. Preferably, the optical axis of the laser outlet 322 is arranged substantially perpendicular to the treatment opening 308, thereby ensuring accurate irradiation of a target scalp region.

In some embodiments, the external laser source may be configured as a fractional laser treatment head, also referred to as a dot-matrix laser source, capable of emitting a plurality of micro-scale laser beams. Within such a laser treatment head, a primary laser beam is generated and subsequently divided into a large number of micro-beams by passing through a microlens array. These micro-beams form numerous microscopic channels or micropores within a stratum corneum layer of the scalp. The micropores extend toward regions surrounding hair follicles. Following the formation of these micropores, a therapeutic medication applied to the scalp can penetrate through the micropores and reach hair follicle roots and dermal layers, thereby bypassing the barrier effect of the stratum corneum and significantly improving medication absorption efficiency.

Referring to FIG. 15, the liquid spraying mechanism includes a spray nozzle 238 configured to communicate with the treatment cavity 306 and to deliver medication toward the scalp. In some embodiments, the liquid spraying mechanism is connected to the treatment cavity 306 through one or more fluid conduits. The liquid spraying mechanism may include a liquid chamber, a micro-pump, and the spray nozzle 238. The spray nozzle 238 is oriented toward an interior region of the treatment cavity 306 so that sprayed medication can be directly applied to the scalp positioned within the treatment opening 308.

The liquid spraying mechanism may be configured to store and deliver therapeutic liquids such as hair regrowth agents, for example, minoxidil-based solutions. During operation, the micro-pump drives the liquid medication from the chamber toward the spray nozzle 238, enabling delivery in an atomized spray form or a directed jet form, thereby allowing precise and controlled administration of medication to the treatment area.

When an edge portion of the treatment opening 308 is brought into contact with the scalp, a sealing engagement is formed between the main body 100 and the scalp at the contact interface. This sealing engagement enables the negative pressure generation mechanism to effectively establish and maintain a negative pressure environment within the treatment cavity 306, thereby improving scalp fixation and enhancing the effectiveness of the treatment process.

In an embodiment, due to the inherent elasticity of human skin, an edge of the treatment opening 308 may be configured as a rigid structure. When the treatment opening 308 is pressed against the scalp, the elastic skin deforms around the rigid edge of the treatment opening 308, thereby forming a sealing engagement between the scalp and the main body 100.

The edge of the treatment opening 308 itself may be formed from an elastic material, enabling the edge to deform together with the scalp when pressed against the scalp surface, thereby forming a sealing interface between the treatment opening 308 and the scalp.

In an embodiment, a flexible sealing member 310 is further provided at the edge of the treatment opening 308. The flexible sealing member 310 is arranged around the periphery of the treatment opening 308 and is configured to form a sealing engagement when the treatment opening 308 is placed in contact with the scalp. The flexible sealing member 310 may have an annular configuration and may be formed from a flexible material, such as silicone, rubber, or other elastomeric materials. The flexible sealing member 310 surrounds the edge of the treatment opening 308 and is fixedly connected to the main body 100. When the treatment opening 308 is positioned against the scalp, the flexible sealing member 310 undergoes elastic deformation and conforms closely to the surface of the scalp, thereby forming a sealed interface between the treatment cavity 306 and the scalp. This sealing arrangement prevents air leakage during the formation of negative pressure and reduces the rigid contact pressure exerted by the device on the scalp.

During operation, a user positions the treatment opening 308 against a target region of the scalp to ensure sealing engagement between the edge of the treatment opening 308 and the scalp surface. Upon activation of the device, the negative pressure generation mechanism is first operated to extract air from the treatment cavity 306, thereby generating a negative pressure environment within the treatment cavity 306. The negative pressure causes the scalp to be drawn toward the treatment opening 308 and stabilized within the treatment cavity 306, thereby tightening the stratum corneum layer of the scalp.

Subsequently, laser energy generated by an external laser source is transmitted through the optical path interface 304 to the laser optical component 320. After optical adjustment by the laser optical component 320, the laser beam is emitted from the laser outlet 322 and directed toward the scalp. The laser beam forms microscopic channels or micropores within the tightened stratum corneum layer of the scalp.

Following the formation of the micropores, the liquid spraying mechanism delivers medication through the spray nozzle 238 into the treatment cavity 306 so that the medication is applied to the micropore region of the scalp. The negative pressure environment further assists in drawing the medication into deeper skin layers, thereby promoting rapid penetration of the medication into the dermis and hair follicle region. Through this coordinated process, the device allows users to perform treatment conveniently without requiring professional operation, thereby simplifying the traditionally complex procedures associated with scalp treatment while maintaining treatment precision and stability. As a result, medication absorption efficiency may be significantly improved while ensuring operational convenience and treatment safety.

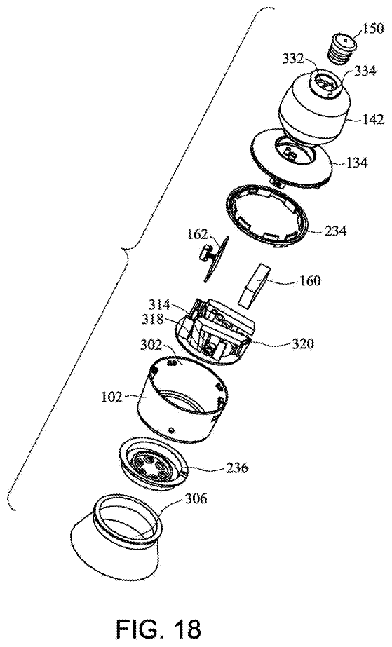

Referring to FIGS. 15 and 18, in an embodiment, the device can further include a dual-chamber arrangement of the liquid storage container 142. The liquid storage container includes a disinfectant chamber 332 and a medication chamber 334.

The disinfectant chamber 332 is configured to store a disinfectant solution, such as medical alcohol, iodine solution, or other antiseptic liquids. The disinfectant chamber 332 is disposed within the liquid storage container 142 and is connected to the spray nozzle 238 through a conduit or pipeline, thereby enabling delivery of disinfectant for disinfecting the scalp before treatment.

The medication chamber 334 is disposed on the shell 102 and is configured to store therapeutic medications used in hair regrowth treatment. The medication chamber 334 is arranged adjacent to the disinfectant chamber 332 within the liquid storage container 142; however, the two chambers are not fluidly connected, thereby preventing cross-contamination between disinfectant and medication. The medication chamber 334 may store therapeutic solutions such as minoxidil-based medications or other hair growth formulations. In some embodiments, the medication chamber 334 can be formed from a transparent or semi-transparent material so that a user can visually observe the remaining amount of medication. The medication chamber 334 is also connected to the spray nozzle 238 through a fluid conduit.

In an embodiment, the disinfectant chamber 332 and the medication chamber 334 are selectively connectable to the spray nozzle 238, enabling the device to deliver either disinfectant or therapeutic medication through the same spray nozzle, depending on the operational stage of the treatment process.

During operation, after the user positions the treatment opening 308 against the scalp and establishes a sealing engagement, the disinfectant chamber 332 is first connected to the spray nozzle 238. The liquid spraying mechanism then delivers disinfectant from the disinfectant chamber 332 through the spray nozzle 238 onto the scalp region to be treated, thereby performing an initial disinfection procedure. After the disinfection process is completed, the fluid connection is switched so that the disinfectant chamber 332 is disconnected and the medication chamber 334 is connected to the spray nozzle 238. Subsequently, the processes of negative pressure adsorption, laser-induced micropore formation, and therapeutic medication spraying are carried out sequentially.

Through this configuration, the device integrates scalp disinfection and medication delivery functions into a single apparatus. As a result, the user is not required to prepare separate disinfection supplies, thereby simplifying the operational procedure associated with conventional treatments. Moreover, the separate storage and selective fluid connection of the disinfectant and therapeutic medication ensure both effective disinfection and safe medication delivery.

In an embodiment, an upper region of the mounting cavity 302 forms a relatively large cavity that is divided by a partition into the disinfectant chamber 332 and the medication chamber 334. The disinfectant chamber 332 and the medication chamber 334 are respectively connected to the treatment cavity 306, which is positioned below the mounting cavity 302, through corresponding pipelines.

The liquid supply mechanism further includes a liquid supply line 336, an atomizing component 252, and a valve assembly 346.

The liquid supply line 336 includes a first pump 338, a second pump 340, a first pipeline 342, and a second pipeline 344. In some embodiments, two spray nozzles 238 are provided. One end of the first pipeline 342 is connected to the disinfectant chamber 332, and the other end is connected to one of the spray nozzles 238. One end of the second pipeline 344 is connected to the medication chamber 334, while the other end is connected to another spray nozzle 238.

The first pump 338 is arranged in association with the disinfectant chamber 332 and is connected to the first pipeline 342 to provide a driving force for delivering the disinfectant. The second pump 340 is arranged in association with the medication chamber 334 and is connected to the second pipeline 344 to provide a driving force for delivering the therapeutic medication. In certain embodiments, the first pump 338 and/or the second pump 340 may be implemented as miniature peristaltic pumps, which provide stable liquid transport while maintaining a compact device structure.

The atomizing component 252 is arranged at the spray nozzle 238 and is configured to atomize and disperse the liquid delivered through the liquid supply line 336. The atomizing component 252 is connected to the corresponding pipeline, such as the first pipeline 342 or the second pipeline 344. In some embodiments, the atomizing component 252 may include an atomizing plate, a flow-guiding structure, or similar atomizing elements. These components convert the incoming liquid, such as disinfectant or therapeutic solution, into fine droplets, thereby preventing the liquid from being discharged in a concentrated column. This atomized spray improves user comfort and increases the effective contact area between the liquid and the scalp surface.

The valve assembly 346 is arranged along the first pipeline 342 and the second pipeline 344 and is configured to control fluid communication within the respective pipelines. The valve assembly 346 may include solenoid valves or mechanical valves that can be selectively actuated by a circuit board 162 of the device. Through the selective opening and closing of the valves, the device can regulate fluid flow through the first pipeline 342 and the second pipeline 344 independently. This configuration ensures that disinfectant and therapeutic medication are delivered through their respective spray nozzles 238 without cross-contamination.

Based on the above structure, during operation of the present embodiment, the pre-disinfection stage is performed first. Specifically, the valve assembly 346 arranged on the first pipeline 342 is opened while the valve assembly 346 arranged on the second pipeline 344 remains closed. Simultaneously, the first pump 338 is activated so that disinfectant stored in the disinfectant chamber 332 is conveyed through the first pipeline 342 to the corresponding spray nozzle 238. Upon passing through the atomizing component 252 disposed at the spray nozzle 238, the disinfectant is atomized into fine droplets and sprayed onto the scalp region to be treated, thereby completing the disinfection procedure.

After the disinfection process is completed, the first pump 338 is stopped, and the valve assembly 346 associated with the first pipeline 342 is closed. Subsequently, the valve assembly 346 of the second pipeline 344 is opened and the second pump 340 is activated. Therapeutic medication stored in the medication chamber 334 is then delivered through the second pipeline 344 to another spray nozzle 238, where the medication is atomized by the atomizing component 252 and sprayed onto the scalp to perform the drug administration process. Through this configuration, the stability and safety of the liquid supply are further improved.

By virtue of the independent dual-pipeline structure, the dual spray nozzles 238, and the separate control of the valve assemblies 346, disinfectant and therapeutic medication are completely separated within the device. This structural separation effectively prevents cross-contamination between the disinfectant and the medication. In addition, positioning the atomizing component 252 at the spray nozzle 238 enables efficient atomization of the liquid, thereby improving adhesion of the sprayed liquid to the scalp surface and enhancing drug absorption. In combination with the precise control of pump operation, the liquid delivery rate can be adjusted according to treatment requirements.

Referring to FIGS. 16 and 17, the laser optical component 320 includes a reflector 324 and a focusing lens 326 arranged sequentially along the optical path. The reflector 324 is configured to redirect laser light received from the optical path interface 304 toward the focusing lens 326, while the focusing lens 326 is configured to focus the laser beam and guide it toward the laser outlet 322.

The reflector 324 is arranged at a position corresponding to the laser emission direction of the optical path interface 304. The mirror surface of the reflector 324 is preset at an appropriate angle to receive the incoming laser beam from the optical path interface 304 and accurately reflect the beam toward the downstream focusing lens 326. This arrangement allows the direction of laser transmission to be flexibly adjusted and facilitates the compact internal layout of optical components within the device.

The focusing lens 326 is disposed downstream of the reflector 324 and upstream of the laser outlet 322 and is substantially coaxial with the optical axis defined between the reflector 324 and the laser outlet 322. The focusing lens 326 may be configured as a curved optical lens capable of refracting and converging the reflected laser beam. Through this focusing action, the laser energy is concentrated into a small focal region before exiting from the laser outlet 322 and acting on the scalp.

In an embodiment, the delivery enhancement mechanism comprises a mist generation mechanism configured to convert the treatment liquid into a mist before delivery into the treatment cavity. The mist generation mechanism includes an atomizing component, such as an atomizing plate or a heating-based atomizer, arranged in communication with the liquid dispensing system. The treatment liquid is transformed into fine mist droplets or aerosolized particles, thereby increasing the surface area of the liquid and enabling more uniform distribution across the scalp. The mist form of the treatment liquid improves contact efficiency and facilitates enhanced absorption into the scalp and surrounding follicular regions.

During operation, laser light emitted from an external laser source enters the device through the optical path interface 304 and is directed toward the reflector 324. After striking the reflector 324 at a preset angle, the laser beam is reflected, and its propagation direction is changed so that it is precisely guided toward the focusing lens 326. As the laser passes through the focusing lens 326, the beam is refracted and concentrated into a highly focused spot. The focused laser beam then exits vertically through the laser outlet 322 and is directed onto the scalp region to be treated, thereby forming micropores in the stratum corneum.

Through the coordinated arrangement of the reflector 324 and the focusing lens 326, the device effectively addresses the challenge of optical path arrangement within the limited internal space of the apparatus. At the same time, the focusing function increases the energy density of the laser beam, enabling the rapid formation of uniform and fine microchannels in the scalp stratum corneum while minimizing damage to surrounding healthy tissue. Furthermore, the stable optical path steering and focusing mechanism eliminates the need for manual adjustment of the laser direction by the user, thereby improving both the ease of operation and the safety of the treatment process.

Referring to FIG. 14, the laser outlet 322 is disposed on the inner wall of the treatment cavity 306, opposite the treatment opening 308. In some embodiments, the laser outlet 322 may be configured as a circular through-hole that is coaxially aligned with the focusing lens 326 of the laser optical component 320. The aperture size of the laser outlet 322 is adapted to correspond to the focused laser spot so as to ensure that the laser beam is emitted completely and accurately.

By arranging the laser outlet 322 opposite the treatment opening 308, the emitted laser beam can be directed straight toward the scalp positioned at the treatment opening without requiring additional optical path adjustments. When the therapy device for stimulating hair growth is in operation, the laser beam focused by the focusing lens 326 exits from the laser outlet 322 and propagates along the central axis of the treatment cavity 306 toward the scalp attached to the treatment opening 308. Because the beam does not pass through other components along this path, laser energy loss is minimized.

Furthermore, after the negative pressure generation mechanism adsorbs and stabilizes the scalp, the laser beam can act more precisely on the tightened stratum corneum region. This configuration reduces positional deviation during micropore formation and ensures that the micropore generation process is carried out accurately and consistently.

The negative pressure generation mechanism includes a negative pressure generator 314, an air extraction pipeline (not shown), and an exhaust port 318.

The negative pressure generator 314 is disposed within the mounting cavity 302 and is configured to generate a controllable suction force for establishing a negative pressure environment within the treatment cavity 306. In some embodiments, the negative pressure generator 314 may be implemented as a miniature vacuum pump, which is characterized by compact size, low operational noise, and stable suction performance.

One end of the air extraction pipeline is connected to an air inlet of the negative pressure generator 314, while the other end extends toward the inner wall of the treatment cavity 306 to form a suction port 316. The air extraction pipeline serves as a gas-conducting channel and may be formed from flexible or rigid tubing capable of maintaining structural integrity under negative pressure conditions. The suction port 316 may be configured as either a single-hole structure or a plurality of holes distributed along the inner wall of the treatment cavity 306. Such a distribution enables a more uniform reduction in pressure within the treatment cavity 306 during suction and prevents the formation of excessive localized negative pressure that could otherwise cause discomfort or damage to the scalp.

Referring to FIGS. 13, 16, and 18, the exhaust port 318 is formed on the shell 102 and communicates with the air outlet of the negative pressure generator 314. The exhaust port 318 functions as a gas discharge channel that allows air expelled by the negative pressure generator 314 to be released to the external environment. In some embodiments, a dust filter may be provided at the exhaust port 318 to prevent external dust or contaminants from entering the interior of the negative pressure generator 314, thereby improving durability and extending the service life of the components.

During operation, once the flexible sealing member forms a sealing engagement with the scalp, the negative pressure generator 314 is activated. Air within the treatment cavity 306 is drawn through the suction port 316 and conveyed through the air extraction pipeline to the air inlet of the negative pressure generator 314. The extracted air is then discharged through the air outlet of the negative pressure generator 314 and expelled through the exhaust port 318 formed in the shell 102. As air is continuously removed from the treatment cavity 306, a stable negative pressure environment is gradually established within the cavity, thereby enabling adsorption and fixation of the scalp and tightening of the stratum corneum.

Through the above configuration, the cooperative arrangement of the air extraction pipeline and the suction port 316 ensures that negative pressure is distributed uniformly within the treatment cavity 306, thereby reducing the risk of localized pressure concentration that could lead to scalp discomfort or injury. In addition, the provision of the exhaust port 318 allows the negative pressure generator 314 to continuously discharge air, thereby improving the stability and efficiency of the negative pressure environment. The user is not required to manually adjust any negative-pressure-related components, and the negative-pressure system operates automatically in coordination with other functional modules of the device. As a result, the operational procedure is simplified while simultaneously enhancing the safety, comfort, and stability of the treatment process.

In an embodiment, referring to FIG. 14, a plurality of openings are provided on the top wall of the treatment cavity 306. Specifically, six openings may be arranged at spaced intervals along a circular pattern. Among these openings, two openings serve as suction ports 316, two openings serve as spray nozzles 238, and two openings serve as laser outlets 322. This symmetrical arrangement facilitates balanced functional distribution within the treatment cavity 306 and contributes to the uniform operation of the negative pressure, liquid spraying, and laser treatment functions.

In an alternative embodiment, instead of providing separate openings for air extraction, liquid delivery, and laser emission, the therapy device can include a single integrated port configured to perform multiple functions. The single port is arranged in communication with the treatment cavity and is configured to selectively or simultaneously facilitate laser light delivery, liquid spraying, and air extraction. In such a configuration, the port can be coupled to a multi-channel conduit or a coaxial passage structure, wherein an optical path for transmitting laser energy, a fluid passage for delivering liquid, and an air passage for air extraction can be arranged within a common outlet.

Referring to FIGS. 15 and 18, the therapy device for stimulating hair growth further includes a contact sensor 348 and the circuit board 162.

The contact sensor 348 is arranged around the treatment opening 308 and is configured to detect the contact status between the flexible sealing member 310 and the scalp. In particular, the contact sensor 348 is disposed adjacent to the mounting region of the flexible sealing member 310 and functions to monitor whether a proper sealing engagement has been established between the treatment opening 308 and the scalp. The contact sensor 348 may be implemented using sensing technologies such as piezoresistive sensing, capacitive sensing, or other suitable contact detection mechanisms. Through such configurations, the contact sensor 348 is capable of continuously monitoring whether the flexible sealing member 310 is properly pressed against the scalp to form a reliable seal. When stable contact is detected, the contact sensor 348 outputs a corresponding electrical signal; conversely, when contact is absent or insufficient, the sensor outputs no signal or an abnormal signal indicating an improper sealing condition.

The circuit board 162 is electrically connected to the contact sensor 348 and the negative pressure generation mechanism, and is configured to control the operation of the negative pressure generation mechanism based on signals received from the contact sensor 348. In some embodiments, the circuit board 162 is integrated on a circuit board disposed within the mounting cavity 302. The circuit board 162 may include components such as a microprocessor, signal processing circuitry, and associated control circuits. Through electrical connections with the contact sensor 348 and the negative pressure generation mechanism, the circuit board 162 is capable of receiving sensor signals, performing signal analysis and logical determination, and outputting corresponding control commands. Based on a preset control program, the circuit board 162 determines whether the contact condition satisfies the required sealing state and accordingly controls the activation or deactivation of the negative pressure generation mechanism. The circuit board 162 may also coordinate the operation of other electrical components within the device to ensure orderly execution of the overall treatment process.

In an embodiment, a battery is arranged within the mounting cavity 302 to supply electrical power to the circuit board 162 and the contact sensor 348. The battery can be configured as a rechargeable battery, and the shell 102 can further be provided with a charging interface to enable external charging of the battery.

In operation, when the user positions the treatment opening 308 against the scalp, the contact sensor 348 continuously monitors the contact condition between the flexible sealing member 310 and the scalp surface. Only when the contact sensor 348 detects a stable sealing engagement and transmits a corresponding valid signal to the circuit board 162 will the circuit board 162 permit activation of the negative pressure generation mechanism. If the sensor detects insufficient contact or loss of sealing, the circuit board 162 prevents the negative pressure generation mechanism from operating. After the negative pressure generation mechanism has established the negative pressure environment and the treatment process is completed, once the contact sensor 348 detects that the device has been removed from the scalp, the circuit board 162 issues a command to stop the operation of the negative pressure generation mechanism.

Through the coordinated operation of the contact sensor 348 and the circuit board 162, the device achieves conditional activation control of the negative pressure generation mechanism. This arrangement prevents operational issues such as failure to establish negative pressure or leakage of air resulting from improper sealing, thereby ensuring stable treatment performance. In addition, the user is not required to manually determine whether an adequate seal has been achieved or to manually control the activation of the negative pressure generation mechanism. Consequently, the operational procedure is simplified, and the risk of incorrect operation is reduced. For home-use scenarios, this intelligent control configuration enhances operational convenience, improves treatment safety, and prevents scalp discomfort that may otherwise occur if negative pressure is applied under inadequate sealing conditions.

Referring to FIGS. 13 and 15, the therapy device for stimulating hair growth further includes a plurality of light-emitting elements 178 for disinfection purposes, disposed within the treatment cavity 306. The light-emitting element 178 is configured to perform ultraviolet disinfection of the scalp before treatment. In addition, the therapy device for stimulating hair growth may further include a flexible sealing member 310 arranged around the edge of the treatment opening 308 to form a sealing engagement when the treatment opening 308 is placed in contact with the scalp.

The light-emitting element 178 is arranged inside the treatment cavity 306, for example, on a top wall of the treatment cavity 306. In some embodiments, the light-emitting element 178 may be implemented as a miniature ultraviolet LED lamp bead, which may be integrated into a corresponding mounting hole formed in the inner wall of the treatment cavity 306. The light-emitting element 178 is configured to emit ultraviolet light for sterilizing and disinfecting the scalp region to be treated before the therapeutic procedure. The flexible sealing member 310, which is configured to form a sealing engagement between the treatment opening 308 and the scalp, has been described in detail above and will not be repeated herein.

In an embodiment, the therapy device is provided only with the light-emitting element 178, without the flexible sealing member 310. In other embodiments, the therapy device for stimulating hair growth can include only the flexible sealing member 310, without the light-emitting element 178. In a further embodiment, the therapy device for stimulating hair growth may include both the light-emitting element 178 and the flexible sealing member 310.

Referring to FIGS. 13 to 17, the device further integrates a laser source. The laser source is configured as an external component independent of the therapy device for stimulating hair growth. The laser source can be implemented as a miniaturized low-power laser generator, such as a semiconductor laser source, which provides stable laser output while maintaining a compact structure that is convenient for portability and storage. The laser source is configured to generate laser radiation used for forming micropores in the stratum corneum of the scalp. The laser source is detachably connected to the optical path interface 304 of the therapy device for stimulating hair growth through a dedicated connecting cable so that laser light generated by the laser source can be transmitted to the laser optical component 320 within the device.

In this configuration, the device employs an external, detachable laser source. The laser source is detachably connected to the optical path interface 304 provided on the main body 100 near the mounting cavity 302, thereby forming a modular structure comprising an external laser source and a treatment cavity. This split configuration reduces the size and weight of the treatment cavity and facilitates independent maintenance, replacement, or upgrading of the laser source.

In an embodiment, to create multiple micropores in the scalp to enhance medication absorption, the laser source may be configured as a fractional laser treatment head, also referred to as a dot matrix laser. Within the laser treatment head, a primary laser beam is generated and subsequently divided into a plurality of micro-beams after passing through a microlens array. These micro-beams form microscopic pores in the stratum corneum of the scalp that are generally invisible to the naked eye. The formed microchannels extend toward the region surrounding the hair follicles, thereby enabling therapeutic medication applied subsequently to penetrate directly toward the hair follicle roots and the dermal layer through the microchannels. By bypassing the barrier function of the stratum corneum, the absorption efficiency of the medication can be significantly improved.

During operation of the therapy device for stimulating hair growth, the user first connects the laser source to the optical path interface 304 of the therapy device for stimulating hair growth. The treatment opening 308 is then placed against the target scalp region. When the contact sensor 348 detects that a proper sealing condition has been established and the circuit board 162 confirms the sealing status, the device initiates the disinfection process. After the disinfection step is completed, the circuit board 162 may issue a control signal to activate the laser source. The generated laser beam is transmitted through the optical path interface 304 to the laser optical component 320 within the device. The laser beam is subsequently redirected by the reflector 324 and focused by the focusing lens 326, after which it exits from the laser outlet 322 positioned opposite the treatment opening 308 and acts on the scalp to create micropores.

Following the formation of the micropores, the device sequentially performs negative pressure adsorption and liquid spraying operations according to a preset control program, thereby completing the overall treatment procedure.

Alternatively, in an embodiment, the laser source can be internally integrated within the shell of the therapy device. In such configurations, the laser source is disposed within the mounting cavity and electrically connected to the circuit board, thereby eliminating the need for an external connection. The internally disposed laser source is aligned with the laser optical component such that laser energy is directly transmitted through the reflector and focusing lens toward the laser outlet. This arrangement provides a more compact and self-contained structure, improves portability, and simplifies operation by removing external attachments, while maintaining effective laser delivery for micropore formation in the scalp.

By combining laser-based micropore formation, controlled negative pressure, and targeted liquid delivery, the device enables enhanced penetration of therapeutic agents into the scalp, thereby improving treatment efficacy compared to conventional approaches.

The coordinated operation of the negative pressure generation mechanism, laser optical component, and liquid spraying mechanism ensures that the scalp is stabilized, conditioned, and treated sequentially and efficiently. The formation of micropores in the stratum corneum facilitates direct delivery of medication to dermal and follicular regions, while the negative pressure environment further enhances absorption and retention of the therapeutic agents.

In an embodiment, the therapy device can also be used for providing therapy to other surfaces apart from the scalp, such as skin on the face, neck, arms, legs, or other body regions where transdermal delivery of therapeutic agents may be beneficial. The phototherapy component, liquid dispensing system, and delivery enhancement mechanism may be configured to operate on various skin surfaces to promote absorption of topical treatments, enhance circulation, or stimulate tissue regeneration. For example, the device may be applied to facial skin for delivering anti-ageing serums or moisturizing agents, or to body skin for administering pain-relief formulations or wound-healing solutions. In such applications, the treatment opening may be adapted in size or shape to conform to the contours of the target treatment area, and the intensity or wavelength of the phototherapy component may be adjusted according to the specific therapeutic requirements of the skin region being treated. The negative pressure generation mechanism, when present, may assist in stabilizing the device against curved or irregular skin surfaces, while the mist generation mechanism may facilitate uniform distribution of the treatment liquid across broader treatment areas.

Additionally, the provision of dual liquid chambers for disinfectant and medication, along with independent pipelines, pumps, and valve assemblies, ensures hygienic operation and prevents cross-contamination. The inclusion of atomizing components enables the uniform distribution of liquids, improving user comfort and treatment consistency. Safety and usability are further enhanced through features such as the contact sensor, intelligent control via the circuit board, and optional sealing members that ensure proper engagement with the scalp before operation.

Moreover, the modular configuration of the laser source, along with the optimized optical path including reflector and focusing lens, allows efficient energy delivery while maintaining a compact structural design. The integration of auxiliary features such as phototherapy light sources, protective covers, and ergonomic structural components further contributes to the versatility and practicality of the device.

Accordingly, the present invention is industrially applicable in the fields of medical devices, dermatology, cosmetology, and personal care equipment manufacturing. The therapy device for stimulating hair growth can be mass-produced using conventional manufacturing techniques and readily integrated into clinical as well as consumer-oriented product lines. The device is suitable for use in dermatology clinics, trichology centers, cosmetic treatment facilities, and home-use applications, providing a versatile solution for scalp treatment and hair regrowth therapies. Owing to its integrated functionalities, including laser-assisted micropore formation, negative pressure stabilization, and controlled liquid delivery, the device enhances the efficiency of topical drug administration and scalp conditioning processes. Furthermore, the modular design, including detachable liquid storage containers and an external or integrated laser source, facilitates ease of maintenance, scalability, and customization according to different treatment requirements. Accordingly, the invention has significant commercial potential and can be widely adopted across the healthcare and personal grooming industries.

Various modifications to these embodiments are apparent to those skilled in the art from the description and the accompanying drawings. The principles associated with the various embodiments described herein may be applied to other embodiments. Therefore, the description is not intended to be limited to the embodiments shown along with the accompanying drawings but is to provide the broadest scope consistent with the principles and the novel and inventive features disclosed or suggested herein. Accordingly, the invention is anticipated to hold on to all other such alternatives, modifications, and variations that fall within the scope of the present invention and appended claims.

1. A therapy device for stimulating hair growth, comprising:
a main body defining a treatment cavity and a treatment opening configured to be placed in contact with a scalp;
a phototherapy component disposed on or within the main body and configured to direct light energy toward the treatment opening;
a liquid dispensing system comprising a liquid storage chamber and at least one liquid outlet in fluid communication with the treatment cavity and configured to deliver a treatment liquid into the treatment cavity; and
a delivery enhancement mechanism configured to enhance penetration, absorption, or distribution of the treatment liquid into the scalp,
wherein the delivery enhancement mechanism comprises at least one of a negative pressure generation mechanism configured to generate a reduced pressure within the treatment cavity or a mist generation mechanism configured to convert the treatment liquid into a mist before delivery, and
wherein the phototherapy component and the delivery enhancement mechanism are configured to operate in coordination to improve delivery of the treatment liquid to the scalp.

2. The device of claim 1, wherein the delivery enhancement mechanism comprises both the negative pressure generation mechanism and the mist generation mechanism.

3. The device of claim 1, wherein the liquid dispensing system comprises at least one of:
a push-actuated dispensing mechanism,
a movable ball dispensing mechanism, or
an air vent-based dispensing mechanism.

4. The device of claim 1, further comprising a contact sensor configured to detect contact between the treatment opening and the scalp and to control operation of the negative pressure generation mechanism based on detected contact.

5. The device of claim 1, further comprising a flexible sealing member disposed around the treatment opening and configured to form an airtight seal with the scalp.

6. A therapy device for stimulating hair growth, comprising:
a main body defining a treatment cavity and a treatment opening configured to be placed in contact with a scalp;
a negative pressure generation mechanism in fluid communication with the treatment cavity and configured to generate a reduced pressure within the treatment cavity;
a laser optical component configured to direct laser energy toward the treatment opening;
a liquid dispensing system configured to deliver a treatment liquid toward the scalp,
wherein the laser optical component is configured to focus laser energy to form a plurality of microchannels in the scalp, and
wherein the negative pressure generation mechanism and the liquid dispensing system are configured to cooperate with the microchannels to facilitate penetration of the treatment liquid into the scalp.

7. The device of claim 6, wherein the laser optical component is configured to receive laser energy from an external laser source through an optical path interface.

8. The device of claim 6, wherein the laser optical component comprises an internal laser source disposed within the main body.

9. The device of claim 6, wherein the laser optical component comprises a reflector configured to redirect laser energy and a focusing lens configured to focus the laser energy toward the treatment opening.

10. The device of claim 9, wherein the focusing lens is positioned at or adjacent to a laser outlet through which the laser energy exits toward the treatment cavity.

11. The device of claim 6, wherein the liquid dispensing system comprises at least one spray nozzle configured to deliver the treatment liquid into the treatment cavity.

12. The device of claim 6, wherein the liquid dispensing system and the laser optical component are configured to deliver treatment liquid and laser energy through a single integrated port opening into the treatment cavity.

13. The device of claim 6, wherein the laser optical component is aligned along an optical axis passing through the treatment cavity toward the treatment opening.

14. The device of claim 6, wherein the negative pressure generation mechanism is configured to draw a portion of the scalp into the treatment cavity before laser irradiation and liquid delivery.

15. A therapy device for stimulating hair growth, comprising:
a main body defining a treatment cavity and a treatment opening configured to be placed in contact with a scalp;
a phototherapy component configured to direct light energy toward the treatment opening;
a liquid dispensing system comprising a liquid storage chamber, a liquid outlet, and a fluid conduit extending between the liquid storage chamber and the liquid outlet; and
a mist generation mechanism configured to convert a treatment liquid into a mist before delivery into the treatment cavity, wherein the phototherapy component and the mist generation mechanism are configured to operate in coordination to improve the distribution and absorption of the treatment liquid on the scalp.

16. The device of claim 15, wherein the liquid dispensing system further comprises a liquid distribution chamber having a smaller volume than the liquid storage chamber, a connection port connecting the liquid storage chamber and the liquid distribution chamber, a sealing head configured to seal the connection port, and a pusher configured to move the sealing head to open the connection port such that a predetermined amount of liquid is transferred from the liquid storage chamber to the liquid distribution chamber.

17. The device of claim 15, wherein the mist generation mechanism comprises an atomizing plate configured to atomize the treatment liquid into fine droplets.

18. The device of claim 15, further comprising a heating element configured to heat the treatment liquid before atomization or delivery.

19. The device of claim 15, wherein the mist generation mechanism comprises an atomizing chamber and an atomizing plate positioned between the atomizing chamber and the treatment cavity.

20. The device of claim 18, wherein the heating element is positioned upstream of the mist generation mechanism such that the treatment liquid is heated before atomization.
